(19) United States
(12) Patent Application Publication
(10) Pub. No.: US 2022/0096510 A1
(43) Pub. Date: Mar. 31, 2022

(54) METHOD AND PHARMACOLOGICAL COMPOSITION FOR THE PREVENTION OF RECURRENT INFECTIONS CAUSED BY CLOSTRIDIUM DIFFICILE

(71) Applicant: UNIVERSIDAD ANDRÉS BELLO, Santiago (CL)

(72) Inventors: Daniel Gonzalo Paredes Sabja, Santiago (CL); Pablo Andrés Castro Córdova, Santiago (CL)

(21) Appl. No.: 17/268,872

(22) PCT Filed: Aug. 17, 2018
(86) PCT No.: PCT/IB2018/056217

§ 371 Date: Feb. 16, 2021

(51) Int. Cl. A61K 31/7048; A61K 31/575; A61K 38/15; A61P 31/04

(52) U.S. Cl. CPC A61K 31/7048; A61P 31/04; A61K 38/15; A61K 31/575

(57) ABSTRACT

Recurrent C. difficile infections are the major cause of death due to C. difficile, which is the causative agent of approximately 20% of antibiotic-associated diarrheas. Conventional treatments for C. difficile infections are not capable of eliminating the rates of recurrence, which occurs in 20-30% of the cases and may be repetitive, the probability of death being greater in each cycle. Until now, the persistence mechanisms of C. difficile for producing recurrence conditions were unknown. In the present invention, we describe the mechanism by which C. difficile spores persist, and a method of treatment with a pharmacological composition based on an antibiotic and nystatin for preventing recurrent C. difficile infections. The route of administration of nystatin is also protected (i.e., oral, intraperitoneal).

The present invention relates to the use of nystatin for the treatment and prevention of recurrent infections produced by Clostridium difficile. The present invention describes a pharmacological composition comprising at least one antibiotic and nystatin for preventing recurrent infections caused by C. difficile. Recurrent C. difficile infections are the major cause of death due to C. difficile, which is the causative agent of approximately 20% of antibiotic-associated diarrheas. Conventional treatments for C. difficile infectious conditions are not capable of eliminating the recurrence rates produced by this pathogen. Recurrence occurs in 20-30% of the cases and may be repetitive, the probability of death becoming greater in each cycle. Until now, the persistence mechanisms of C. difficile for producing recurrence conditions are unknown and the present invention proposes the mechanism by which C. difficile spores persist in the body, and describes a method of treatment for recurrent infections produced by C. difficile. A method of treatment for recurrent infections produced by C. difficile through the use of a combination of drugs comprising nystatin is described.

### DESCRIPTION OF WHAT IS KNOWN IN THE ART

One of the most frequent intrahospital infections is caused by the anaerobic spore-forming enteropathogen Clostridium difficile, which has been established to be the causative agent of 20 to 30% of diarrheas associated to antibiotic treatments. Traditionally, C. difficile infections (CDI) have been associated to hospitalized patients on antibiotic treatment. However, there has recently been reported an increase in the CDI cases acquired in the community worldwide. In general, manifestations of CDI vary from mild diarrheas without systemic manifestations, to conditions characterized by fulminant colitis with toxic megacolon and perforations in the colon tract. Standard antibiotic treatment for CDI comprises orally administered vancomycin and/or metronidazole, achieving favorable results and mortality rates ranging from 1 to ˜5% (Evans and Safdar, 2015). However, the main clinical challenge of CDI is that 30% of the patients with a first episode of CDI, exhibit a second episode of CDI. This is aggravated by the fact that the probabilities of presenting a third and a fourth episode of recurrent CDI (R-CDI) increase by 40 and 65%. Likewise, mortality rates can also increase to above 30% after several recurrence episodes. This clinical challenge has focused efforts on developing new drugs and therapeutic strategies seeking to prevent R-CDI, but scarce success has been obtained or massification has been elusive. Therefore, the high CDI recurrence rate and the reduced spectrum of available therapies represent a great opportunity for the development of new formulations to address key aspects of the pathogenesis of R-CDI.

The major cause that directly contributes to the existence of R-CDI is that, during the infective cycle, this anaerobic enteropathogen, in parallel with the production of the major virulence factors, enterotoxin TcdA and cytotoxin TcdB, responsible for the clinical symptoms of the infection, initiates the production of new spores.

Antibiotic therapies for treating CDI consisting of metronidazole and vancomycin are another factor that contributes to the elevated R-CDI rates, mainly because they help maintain a dysbiosis status of the microbiota (Deakin et al., 2012), but more importantly, because metronidazole and vancomycin do not inhibit spore production (Baines et al., 2009). The relevance of the sporulation process was demonstrated by Deakin et al. (2012), who used a murine R-CDI model to prove that spo0A mutant strains, deficient in spore formation, do not cause R-CDI (Deakin et al., 2012), indicating that formation and persistence of C. difficile spores is key for R-CDI.

In this sense, previous in vitro studies by the inventors have demonstrated that C. difficile spores efficiently adhere to intestinal epithelial cells in vitro (i.e., adherence rates >70% of total spores) (Paredes-Sabja and Sarker, 2012), suggesting that the adherence of the spores contributes to the persistence thereof in the colon tract.

On the other hand, it is known that C. difficile spores germinate only in the presence of primary bile salts (i.e., taurocholate or cholate) and the co-germinant L-glycine, which are present in the colonic lumen (Sorg and Sonenshein, 2008; Giel et al., 2010; Theriot et al., 2014).

### How the Problem of Recurrent Infections has been Addressed Previously

In 2010, the Society for Healthcare Epidemiology of America (SHEA) published an update of protocols of clinical approach to R-CDI, which are based on the treatment with the same antibiotics used for treating the first episode of CDI. SHEA's recommendations for treating R-CDI are: i) in the case of a mild to moderate CDI, discontinuation of the causative antibiotic is often enough; ii) if this measure does not succeed, the treatment of choice is oral metronidazole for 10 to 14 days; iii) in cases of uncomplicated serious CDI, oral vancomycin for 10 to 14 days is recommended as first-line therapy; iv) for complicated serious CDI, vancomycin is generally administered either orally or by nasogastric tube combined with intravenous metronidazole. In the case of a second R-CDI, the first-line treatment corresponds to oral vancomycin for prolonged periods and with progressive reduction for 2 to 8 weeks. The rationale of this scheme is to avoid the appearance of vegetative forms of C. difficile while normal colon microbiota is being restored. Treatment for subsequent episodes of R-CDI becomes a challenge as a result of the permanent dysbiosis status of the microbiota, the presence of spores and the scarce number of alternative therapies for treating CDI. In this sense, SHEA provides recommendations for R-CDI which are based on low-quality evidence, shedding some light on how poorly studied are the alternative therapies for treating R-CDI.

The main problem of the therapies based on vancomycin and metronidazole is that they generate a dysbiosis status in colon microbiota, reducing the presence of other members of the resident flora such as Bacteroides spp., other Clostridia, Fusobacterium spp and Bifidiocterium spp. Some of these enterotypes inhibit the growth of C. difficile and hinder germination through metabolization of the bile salts when they are present in a “normal microbiota” status. On the contrary, a dysbiotic microbiota favors both the germination of C. difficile spores and the onset of a clinical condition such as recurrence (Theriot et al., 2014).

According to these antecedents, it is evident that recurrent C. difficile infections (R-CDI) are based mainly on two points: i) the presence of spores in the colon tract of the host (Deakin et al., 2012); and ii) a permanent dysbiosis status of the microbiota (Theriot et al., 2014). Thus, the scientific community has focused on understanding the relevant aspects to be able to develop therapies for attacking these two factors.

Regarding C. difficile spores, the effect of different antibiotics on the decrease in sporulation, toxin production and growth of germinated spores has been investigated. For example, an in vitro study demonstrated that ramoplanin, a non-adsorbable, glycopeptidic antibiotic (US 20080113902 A1), unlike metronidazole and vancomycin, adheres to the exosporium of C. difficile spores and acts on the cell once the C. difficile spore has germinated (Kraus, Lyerly and Carman, 2015). Unfortunately, clinical studies demonstrate that this antibiotic has an effectiveness in the resolution of the clinical condition and the prevention of recurrence that is similar to vancomycin, whereby it could be considered as a mere technical alternative to the state of the art. Studies performed with tigecyclin, an antibiotic of the glycylcycline family that inhibits protein synthesis by reversible binding to the 30S subunit of the ribosome, decreases the efficiency of spore and toxin production down to sub-inhibitory concentrations (Aldape et al., 2015). However, clinical studies are required to determine its efficiency in the treatment of CDI and R-CDI. On the other hand, rifaximin is a non-absorbed antibiotic, with demonstrated in vitro activity against almost all C. difficile strains. However, a critical amino acid substitution in the β subunit of RNA polymerase leads to high resistance rates. Generally, rifaximin is used after vancomycin in the treatment of R-CDI, although with an efficiency rate of only 64%. However, these strategies are unifactorial since they only affect the vegetative form of C. difficile, having no activity against C. difficile spores.

In respect of the microbiota, recent studies have demonstrated that metabolization of bile salts by defined species of the colon microbiota is key in the resistance or susceptibility of the host to CDI and R-CDI (Theriot et al., 2014). The presence of non-pathogenic Clostridium species capable of 7α-dehydroxylating primary bile salts (i.e., cholates and kenodeoxycholate), reduces the bioavailability of the taurocholate germinant producing an increase in secondary bile salts (i.e., deoxycholate and lithiocholate). An increase in deoxycholate via 7α-dehydroxylation allows to trigger germination of C. difficile spores for a subsequent inactivation of the vegetative cell by this same bile salt (Sorg and Sonenshein, 2008). On the contrary, the absence of these species, as a result of antibiotic therapy, contributes to the increase in primary bile salts, especially of the taurocholate germinant, and to a colon environment favorable for germination and proliferation of the C. difficile spores produced during the first infection cycle (Theriot et al., 2014). Although the intention of these studies was to identify key components of the microbiota for the development of a bacteriotherapy, the complexity of the interactions between the microbiota and different aspects of the host (i.e. immune system, physiology, metabolism and nervous system) and the lack of knowledge about these interactions, result in that it would be irresponsible to suggest bacteriotherapies based on specific species or a combination of species of the normal flora, which could trigger autoimmune disorders, colon cancer, kidney stones and metabolic alterations. Further holistic studies are necessary to determine the long-term effects of bacteriotherapies on the host, rendering its immediate use unfeasible.

As mentioned above, the existing solutions for treating CDI are not sufficient to treat R-CDI. Possibly, the only alternative of antibiotic treatment that could reduce the occurrence rate of R-CDI is fidaxomicin, approved by the FDA in 2011 for the treatment of severe CDI. In vitro studies have demonstrated that fidaxomicin inhibits RNA synthesis, and reduces production and growth of C. difficile spores. However, the results of a phase III clinical trial were not as surprising. Results demonstrate that, for late R-CDI (i.e., after 28 days), 35.5% of patients treated with vancomycin and 19.7% of patients treated with fidaxomicin presented R-CDI. In the case of early recurrences, 27% of patients treated with vancomycin and 8% of patients treated with fidaxomicin presented R-CDI. Even though fidaxomicin achieves a reduction of R-CDI episodes, these continue to be present in a significant percentage of the patients. The above, together with the elevated cost of fidaxomicin, renders its use unattractive.

Although efforts have been made to implement a therapy with probiotics (S. boulardii, Lactobacillus plantarum 299v and Lactobacillus GG) after finishing conventional therapy, these have not been successful. In this sense, the only randomized study that yielded results close to statistical significance was one which evaluated vancomycin during 10 days followed by S. boulardii during 4 days.

Another therapeutic strategy for the treatment of CDI and R-CDI is passive immunization with human IgG1 antibodies specific for toxins TcdA (actoxumab) and TcdB (bezlotoxumab). In phase III clinical trials, it was observed that passive immunization with bezlotoxumab antibodies to patients with CDI is sufficient to reduce R-CDI. This treatment reduces R-CDI rates to 17% as compared with the 27% observed in those patients treated with vancomycin. This therapy has been approved by the FDA, but the costs associated to this passive immunotherapy are high.

The most successful alternative for treating R-CDI is the restoration of microbiota diversity so as to inhibit the growth of C. difficile, through fecal microbiota transplant (FMT). FMT comprises administering fecal content from a healthy individual to the patient in order to restore protective intestinal microbiota. Recent phase 2 clinical trials (NCT02299570) demonstrated that a formulation of fecal microbiota achieved 87.1% efficacy in the resolution of clinical conditions by C. difficile.

Various drug patents were found describing the treatment for CDI. Application WO 2001035983 mentions a pharmaceutical composition based on cysteine and cysteine derivatives for the treatment of diarrhea caused by C. difficile, wherein it was demonstrated that 10 mM cysteine reduces toxin production in vitro and suggests that it could prevent CDI. Patent applications CA 2779413 A1, US 20110280847, WO 2010062369 A2 mention methods and bile salt formulations for inhibiting germination and growth of C. difficile. Rineh et al. (2014) discloses inhibiting spore germination for treating CDI. However, these references do not disclose the inhibition of internalization of bacterial spores. U.S. Pat. No. 5,773,000 A refers to the use of specific antibodies for C. difficile and its toxins in combination with vancomycin, bacitracin or metronidazole.

On the other hand, within the prior art related to the use of nystatin, mention can be made, for example, of patent application WO 2007023370 which refers to the use of nystatin for the treatment of malaria. Patent application WO 2003017960 describes a mouthwash based on metronidazole and nystatin for the prevention of oral bacterial and fungal infections. Patent application EP1261351 mentions a reduced-toxicity formulation based on nystatin to be administered parenterally for the treatment of systemic fungal infections. Buzyn et al (1999) discloses the use of a combination of gentamicin, vancomycin and nystatin for total gut decontamination in immunocompromised patients. However, this reference does not specifically disclose treating or preventing recurrent CDI. In summary, in the state of the art there do not exist patent applications associated to the use of nystatin for the treatment of CDI and R-CDI.

Thus, from the reading of the related literature, it can be deduced that a formulation comprising nystatin for the treatment of R-CDI has not been anticipated in the state of the art. In addition, R-CDI cases constitute a technical problem that has not been efficiently solved.

The present invention has determined that spores adhered to intestinal epithelial cells in vitro are endocytosed. Endocytosed spores are naturally isolated from germinants such as primary bile salts (for example, taurocholate or cholate) and from the co-germinant L-glycine, which are present in the colonic lumen. For this reason, the above mentioned intracellular spores can remain metabolically inactive. Therefore, these intracellular spores would be resistant to antibiotic treatments and to the action of enzymes. These observations suggest that part of the C. difficile spores that interact with the colon epithelium are persisting intracellularly in the host, and once released to the colon, would be the causative agents of the R-CDI episodes. Thus, according to the antecedents disclosed, we can indicate that endocytosis of spores by intestinal epithelial cells appears to be a critical stage for the development of recurrence.

The present invention describes that nystatin, a cholesterol sequestrant, efficiently inhibits endocytosis of C. difficile spores. Therefore, a treatment combining nystatin (an inhibitor of spore endocytosis)+taurocholate that promotes germination of C. difficile spores+an antibiotic that eliminates vegetative cells (for example, vancomycin and/or ramoplanin) could allow the spores to avoid being endocytosed, remaining exposed to the germinants naturally existing in the intestine in dysbiosis status and additionally within the formulation. Germinants will trigger spore germination with the consequent decrease of dormant (antibiotic-resistant) spores and will increase the number of (antibiotic-sensitive) vegetative cells, allowing vancomycin and/or ramoplanin to efficiently eliminate vegetative cells and germinated spores of C. difficile. Since spores are the source of R-CDI, the combination of nystatin+antibiotics plausibly contributes to reduce these recurrence conditions.

Results of the examples show that the combination of nystatin and an antibiotic, for example vancomycin, and taurocholate, or else a combination of vancomycin, ramoplanin and nystatin, significantly reduces the presentation of diarrhea and clinical symptoms associated to CDI. Therefore, the present invention describes a composition oriented to decreasing the spore load, favoring germination, thus allowing antibiotics to eliminate these new vegetative cells and, consequently, reduce the incidence of R-CDI.

For these reasons, we believe that a treatment oriented to reducing R-CDI should have a multifactorial approach which should: i) decrease endocytosis of spores (for example, with nystatin), ii) favor germination thereof by leaving them exposed to the germinants existing in the colonic lumen such as taurocholate, and adding more taurocholate and iii) finally destroy the germinated and vegetative state spores (for example, with an antibiotic treatment such as vancomycin and/or ramoplanin).

### OTHER REFERENCES

Aldape, M. J. et al. (2015) ‘Tigecycline suppresses toxin A and B production and sporulation in Clostridium difficile’, Journal of Antimicrobial Chemotherapy, 70(1), pp. 153-159.
Baines, S. D. et al. (2009) ‘Activity of vancomycin against epidemic Clostridium difficile strains in a human gut model’, Journal of Antimicrobial Chemotherapy, 63, pp. 520-525.
Brophy, K. et al. (2010) ‘Clinical drug therapy for Canadian Practice’ (2^{nd} Edition) https://books.google.cl/books?id=xi8c-EBVkV8C&printsec=frontcover&hl=es#v=onepage&q&f=false
Buzyn, A. et al. (1999) “Reflections on gut decontamination in hematology” Clin Microbiol Infect, 5, pp 449-456.
Deakin, L. J. et al. (2012) ‘The Clostridium difficile spo0A gene is a persistence and transmission factor.’, Infection and immunity, 80(8), pp. 2704-2711.
Evans, C. T. and Safdar, N. (2015) ‘Current Trends in the Epidemiology and Outcomes of Clostridium difficile Infection’, Clinical Infectious Diseases, 60(suppl 2), pp. S66-S71.
Giel, J. L. et al. (2010) ‘Metabolism of Bile Salts in Mice Influences Spore Germination in Clostridium difficile’, PLoS ONE. Edited by A. J. Ratner. Public Library of Science, 5(1), p. e8740.
Kraus, C. N., Lyerly, M. W. and Carman, R. J. (2015) ‘Ambush of Clostridium difficile spores by ramoplanin: Activity in an in vitro model’, Antimicrobial Agents and Chemotherapy, 59(5), pp. 2525-2530.
Paredes-Sabja, D. and Sarker, M. R. (2012) ‘Adherence of Clostridium difficile spores to Caco-2 cells in culture’, Journal of Medical Microbiology, pp. 1208-1218.
Rineh, A. et al. (2014) ‘Clostridium difficile infection: molecular pathogenesis and novel therapeutics” Expert Rev. Anti-infect, Ther. 12(1), pp. 1-20.
Sorg, J. A. and Sonenshein, A. L. (2008) ‘Bile salts and glycine as cogerminants for Clostridium difficile spores’, Journal of Bacteriology, 190, pp. 2505-2512.
Sun, X. et al. (2011) ‘Mouse relapse model of Clostridium difficile infection’, Infection and Immunity, 79, pp. 2856-2864.
Theriot, C. M. et al. (2014) ‘Antibiotic-induced shifts in the mouse gut microbiome and metabolome increase susceptibility to Clostridium difficile infection.’, Nature communications, 5, p. 3114.

### DESCRIPTION OF THE INVENTION

The present invention refers to a formulation for treating recurrent C. difficile infections in a subject or for preventing the risk of developing a recurrent C. difficile infection in a subject, which comprises an agent that inhibits internalizing of C. difficile spores.

The present invention refers to a formulation for treating recurrent C. difficile infections or for preventing the risk of developing a recurrent C. difficile infection in a subject, wherein the formulation comprises an agent that inhibits internalizing of C. difficile spores such as nystatin, chlorpromazine or indomethacin, or chemical or biological analogs thereof.

The present invention refers to a formulation for treating or preventing the risk of developing recurrent C. difficile infections in a subject, wherein the formulation comprises one or more antibiotics having activity against C. difficile and an agent that inhibits internalization of C. difficile spores, wherein the one or more antibiotics having activity against C. difficile are selected from the group consisting of vancomycin, ramoplanin, metronidazole, fidaxomicin, rifaximin and tigecyclin. Preferably, the one or more antibiotics having activity against C. difficile are selected from vancomicyn and ramoplanin.

The present invention refers to a formulation for treating or preventing the risk of developing recurrent C. difficile infections in a subject in need of thereof, wherein the formulation comprises one or more a antibiotics having activity against C. difficile selected from vancomycin and an agent that inhibits internalization of C. difficile spores selected from nystatin.

The present invention refers to a formulation comprising one or more antibiotics having activity against C. difficile, an agent that inhibits internalization of C. difficile spores and a spore germinant belonging to primary bile salts, for example taurocholate or cholate, for treating or preventing the risk of developing a recurrent C. difficile infection in a subject.

The present invention refers to a formulation comprising one or more antibiotics having activity against C. difficile and an agent that inhibits internalization of C. difficile spores for treating or preventing recurrent C. difficile infections in a subject, wherein the recurrent C. difficile infection is C. difficile colitis or wherein the recurrent C. difficile infection is pseudomembranous colitis.

The formulation of the present invention further comprises a pharmaceutically acceptable solvent or carrier. The formulation of the present invention further comprises one or more pharmacologically acceptable excipients. The formulation of the invention is manufactured in the form of syrup, capsules, serum, granules, encapsulated in nanoparticles.

In the formulation of the invention the one or more antibiotics having activity against C. difficile and the agent that inhibits internalization of C. difficile spores have a weight ratio of about 40:1 to about 3:1. Preferably, a weight ratio of about 9:1 or about 4:1.

In the formulation of the invention the one or more antibiotics having activity against C. difficile are present in an amount that ranges from about 80-100 mg per day to about 4 g per day, wherein the minimum dose of 80-100 mg per day corresponding to the minimum oral dose of antibiotic for the treatment of infantile infections considering a minimum body weight of 2 kg, and wherein the maximum dose of 4 g per day corresponding to the maximum oral dose of antibiotic for the treatment of adult infections (Brophy et al., 2010).

In the formulation of the invention the one or more antibiotics having activity against C. difficile are present in an amount that ranges from about 100 mg to about 4 g per day. In the formulation of the invention the one or more antibiotics having activity against C. difficile are present in an amount of about 50 mg/kg/day.

In the formulation of the invention the one or more antibiotics having activity against C. difficile are present in a minimum dose of 40-50 mg/kg/day which corresponds to the minimum oral dose of antibiotic for the treatment of infantile infections considering a minimum body weight of 2 kg.

In the formulation of the invention the one or more antibiotics having activity against C. difficile are present in a maximum dose of 1 g/kg/day which corresponds to the maximum oral dose of antibiotic for the treatment of infantile infections considering a minimum body weight of 2 kg.

In the formulation of the invention the agent that inhibits internalization of C. difficile spores is present in an amount that ranges from about 100,000 UI to about 3,000,000 UI per day. In formulation of the invention the agent that inhibits internalization of C. difficile spores is present in an amount that ranges from about 4,250 UI/kg to about 17,000 UI/kg.

In one embodiment, the invention refers to the use of a formulation comprising an agent that inhibits internalization of C. difficile spores, which serves for preparing a medicament useful for treating R-CDI or for preventing the risk of developing R-CDI in a subject.

In one embodiment, the invention refers to the use of nystatin as an agent that inhibits internalization of C. difficile spores for treating R-CDI or for preventing the risk of developing R-CDI in a subject.

In one embodiment, the invention refers to the use of an effective amount of one or more antibiotics having activity against C. difficile and an agent that inhibits internalization of C. difficile spores for the manufacture of a first medicament comprising the one or more antibiotics and a second medicament comprising the agent that inhibits internalization of C. difficile spores for treating or preventing the risk of developing recurrent C. difficile infections in a subject in need thereof.

In one embodiment, the invention refers to the use of an effective amount of one or more antibiotics having activity against C. difficile in the manufacture of a medicament for treating or preventing the risk of developing recurrent C. difficile infections in a subject in need thereof, wherein the one or more antibiotics are used in combination with an agent that inhibits internalization of C. difficile spores.

In one embodiment, the invention refers to the use of an effective amount of an agent that inhibits internalization of C. difficile spores in the manufacture of a medicament for treating or preventing the risk of developing recurrent C. difficile infections in a subject in need thereof, wherein the agent that inhibits internalization of C. difficile spores is used in combination with one or more antibiotics having activity against C. difficile.

In the use of the invention the one or more antibiotics having activity against C. difficile selected from the group consisting of vancomycin, metronidazole, ramoplanin, fidaxomicin, rifaximin, tigecyclin, and the agent that inhibits internalization of C. difficile spores comprises nystatin or chemical or biological analogs of nystatin, wherein the use of invention is for treating or preventing the risk of developing recurrent C. difficile infections in a subject in need thereof, wherein the recurrent C. difficile infection is C. difficile colitis or wherein the recurrent C. difficile infection is pseudomembranous colitis.

In the use of the invention the one or more antibiotics having activity against C. difficile is selected from vancomycin and the agent that inhibits internalization of C. difficile spores is nystatin, wherein the use further comprises a spore germinant such as taurocholate.

In one embodiment, the invention relates to a method for treating or preventing the risk of developing recurrent C. difficile infections in a subject in need thereof, which comprises administering to the subject an effective amount of one or more antibiotics having activity against C. difficile and an agent that inhibits internalization of C. difficile spores. In the method of the invention the antibiotic is selected from vancomycin, ramoplanin, metronidazole, fidaxomicin, rifaximin. The agent that inhibits internalization of C. difficile spores is nystatin. Preferably, the one or more antibiotics are selected from vancomycin and ramoplanin, and the R-CDI is C. difficile colitis or the R-CDI is pseudomembranous colitis.

In one embodiment, the invention relates to a composition comprising one or more antibiotics having activity against C. difficile for use in a method for treating or preventing the risk of developing recurrent C. difficile infections in a subject in need thereof, wherein the subject is also administered an agent that inhibits internalization of C. difficile spores.

In one embodiment, the invention relates to a composition comprising an agent that inhibits internalization of C. difficile spores for use in a method for treating or preventing the risk of developing recurrent C. difficile infections in a subject in need thereof, wherein the subject is also administered one or more antibiotics having activity against C. difficile.

In one embodiment, the invention relates to a composition comprising one or more antibiotics having activity against C. difficile and an agent that inhibits internalization of C. difficile spores for use in a method for treating or preventing the risk of developing recurrent C. difficile infections in a subject in need thereof. Wherein the recurrent C. difficile infection is C. difficile colitis, and wherein the C. difficile colitis is pseudomembranous colitis and wherein the subject is a mammal.

In the composition of the invention the one or more antibiotics are selected from the group consisting of vancomycin, ramoplanin, metronidazole, fidaxomicin and rifaximin. Preferably, the one or more antibiotics comprise vancomycin and/or ramoplanin. More preferably, the one or more antibiotics comprise vancomycin.

In the composition of the invention the agent that inhibits internalization of C. difficile spores is nystatin, and further comprising a spore germinant, such as taurocholate.

The composition of the invention further comprising a pharmaceutically acceptable solvent or carrier. The composition of the invention further comprising one or more pharmacologically acceptable excipients. The composition of the invention is manufactured in the form of syrup, capsules, serum, granules, encapsulated in nanoparticles.

In the composition of the invention the one or more antibiotics having activity against C. difficile and the agent that inhibits internalization of C. difficile spores have a weight ratio of about 40:1 to about 3:1. Preferably, about 9:1 or about 4:1.

In the composition of the invention the one or more antibiotics having activity against C. difficile are present in an amount that ranges from about 100 mg to about 4 g per day. In the composition of the invention the one or more antibiotics having activity against C. difficile are present in an amount of about 50 mg/kg/day.

In the composition of the invention the agent that inhibits internalization of C. difficile spores is present in an amount that ranges from about 100,000 UI to about 3,000,000 UI per day. In the composition of the invention the agent that inhibits internalization of C. difficile spores is present in an amount that ranges from about 4,250 UI/kg to about 17,000 UI/kg.

In one embodiment, the invention comprises a kit comprising one or more antibiotics having activity against C. difficile and a package insert comprising instructions for using the one or more antibiotics in combination with an agent that inhibits internalization of C. difficile spores for treating or preventing the risk of developing recurrent C. difficile infections in a subject in need thereof.

In one embodiment, the invention comprises a kit comprising one or more antibiotics having activity against C. difficile and an agent that inhibits internalization of C. difficile spores and a package insert comprising instructions for using the one or more antibiotics and the agent that inhibits internalization of C. difficile spores for treating or preventing the risk of developing recurrent C. difficile infections in a subject in need thereof.

In one embodiment, the invention comprises a kit comprising an agent that inhibits internalization of C. difficile spores and a package insert comprising instructions for using the agent that inhibits internalization of C. difficile spores in combination with one or more antibiotics having activity against C. difficile for treating or preventing the risk of developing recurrent C. difficile infections in a subject in need thereof.

The kit of the invention comprises one or more antibiotics selected from the group consisting of vancomycin, ramoplanin, metronidazole, fidaxomicin and rifaximin. Preferably, the one or more antibiotics comprise vancomycin and/or ramoplanin. More preferably, the one or more antibiotics comprise vancomycin. And the recurrent C. difficile infection is C. difficile colitis, wherein the C. difficile colitis is pseudomembranous colitis.

The kit of the invention comprises an agent that inhibits internalization of C. difficile spores which is nystatin, and the kit of the invention further comprising a spore germinant, such as taurocholate.

### BRIEF DESCRIPTION OF THE FIGURES

FIG. 1 shows transmission electron micrographs of polarized T84 (FIG. 1A) and Caco-2 (FIG. 1D) cells. T84 and Caco-2 cells were infected with vegetative cells and spores of C. difficile strains 630 and R20291, respectively. FIGS. 1B and 1C correspond to enlargements of FIG. 1A, showing intracellular spores. FIGS. 1E and 1F correspond to enlargements of FIG. 1D. Scale bars represent 1 μm.

FIG. 2 shows a confocal microscopy microphotograph of a small intestine segment and a colon segment of C57BL/6 mice infected with C. difficile spores. FIG. 2A is colon and FIG. 2B corresponds to small intestine. Images are representative of 3 different sites, analyzed in 3 different mice. Arrows indicate spores that are within the tissue. Scale bar corresponds to 10 μm.

FIG. 3 shows the effect of nystatin on the inhibition of endocytosis of C. difficile spores in Caco-2 cells. FIG. 3A shows the internalization of C. difficile spores at different nystatin concentrations. FIG. 3B shows the adherence of C. difficile spores at different nystatin concentrations. Cells were treated with increasing nystatin concentrations for 1 hour and then infected during 3 hours, in the presence of nystatin, with a multiplicity of infection (MOI) of 10 of C. difficile R20291 spores pre-incubated for 1 hour with fetal bovine serum (FBS).

FIG. 4 shows the effect of nystatin on the inhibition of endocytosis of C. difficile spores in T84 cells. FIG. 4A shows that internalization of C. difficile spores is inhibited by nystatin. FIG. 4B shows that adherence of C. difficile spores is not interfered with by nystatin. T84 cells were infected with the highest nystatin concentration (30 μM) used in the Caco-2 cells of FIG. 3. Cells were treated with nystatin for 1 hour and then infected during 3 hours, in the presence of nystatin, with MOI 10 of C. difficile R20291 spores pre-incubated for 1 hour with FBS.

FIG. 5 shows the effect of nystatin on the reduction of taurocholate-resistant C. difficile spores in infected Caco-2 cells. Caco-2 cells at 2 days of confluence were treated with 30 μM nystatin for 1 h and then infected with C. difficile spores at MOI 10 during 3 h. Cells were then washed, treated with 0.1% taurocholate for germinating the spores exposed to taurocholate (extracellular) and then treated with 70% ethanol to kill the ethanol-sensitive vegetative cells. Cells were macerated and seeded onto BHIS plates, supplemented with 0.1% taurocholate and incubated for 2 days in anaerobic conditions. Bars indicate the percentage of CFU of ethanol-resistant spores for the different treatments.

FIG. 6 shows a scheme of experimental infection design wherein nystatin is administered intraperitoneally. All mice were treated with a mixture of antibiotics for 3 days, after 2 days they were treated with clindamycin, on the following day they were infected with 1×10⁷ C. difficile spores, they were evaluated for 2 days when the initial infection manifests itself and then treated with i) oral vancomycin 50 mg/kg and intraperitoneal (I.P.) intralipids (n=7) and ii) vancomycin 50 mg/kg with I.P. nystatin 12,000 IU/kg in intralipids (n=6) for 7 days. After finishing the treatment, the mice were evaluated on a daily basis until day 16 when they were sacrificed.

FIG. 7 shows the daily average weight of mice treated with vancomycin 50 mg/kg (white circles) and treated with vancomycin 50 mg/kg plus nystatin 12,000 IU/kg (black triangles), as indicated in FIG. 6.

FIG. 8 shows the percentage of mice which manifested diarrhea throughout the experiment. Indicated are mice treated with vancomycin 50 mg/kg (white circles) and treated with vancomycin 50 mg/kg plus nystatin 12,000 IU/kg I.P. (black triangles), as indicated in FIG. 6. It can be observed that between days 10 and 16, the percentage of mice presenting diarrhea was lower in mice treated with vancomycin and nystatin in comparison to mice treated only with vancomycin.

FIG. 9 shows the diarrhea score observed on days 2 and 12 post infection with C. difficile spores in mice treated with vancomycin 50 mg/kg or vancomycin 50 mg/kg and nystatin 12,000 IU/kg I.P., as indicated in FIG. 6. FIG. 9A shows the distribution and average value of diarrhea score observed on day 2. FIG. 9B shows distribution and average value of the diarrhea score observed on day 12. Results are shown as mean±SEM (Standard Error of the Mean). Asterisk indicates P<0.05.

FIG. 10 indicates the time taken by mice infected by C. difficile and treated with vancomycin 50 mg/kg or vancomycin 50 mg/kg and nystatin 12,000 IU/kg I.P. (as indicated in FIG. 6) to present diarrhea associated to a recurrent C. difficile infection.

FIG. 11 shows the CFU load of C. difficile spores in stools that were collected daily. Dotted line indicates limit of detection. Indicated are mice treated with vancomycin 50 mg/kg (white circles) and treated with vancomycin 50 mg/kg plus nystatin 12,000 IU/kg (black triangles), as indicated in FIG. 6. No significant differences were observed between the treatments.

FIG. 12 shows a scheme of experimental infection design in which nystatin is administered orally at a concentration of 8,500 IU/kg, starting from one day before infection with C. difficile spores, and then vancomycin 50 mg/kg once the clinical conditions of CDI have manifested themselves. For this purpose, all mice were treated with a mixture of antibiotics during 3 days, after 2 days they were treated with clindamycin, on the following day they were infected with 1×10⁷ C. difficile spores, they were evaluated for 2 days when the initial infection manifests itself, and then treated with vancomycin during 5 days. After finishing the treatment with vancomycin, mice were evaluated daily until day 12 when they were sacrificed. The groups evaluated were i) untreated infected mice (n=4); ii) mice treated with nystatin 8,500 IU/kg (n=4); iii) mice treated with vancomycin 50 mg/kg (n=5); and iv) mice treated with vancomycin 50 mg/kg and nystatin 8,500 IU/kg (n=5).

FIG. 13 shows the average weight throughout the experiment indicated in FIG. 12. Indicated are untreated mice (white triangles), mice treated with nystatin 8,500 IU/kg (grey diamonds), mice treated with vancomycin 50 mg/kg plus nystatin 8,500 IU/kg (white squares) and mice treated with vancomycin 50 mg/kg (white circles).

FIG. 14 shows the average percentage of mice with diarrhea. Indicated are untreated mice (white triangles), mice treated with nystatin 8,500 IU/kg (diamonds), mice treated with vancomycin 50 mg/kg plus nystatin 8,500 IU/kg (squares) and mice treated with vancomycin 50 mg/kg (circles). Treatments were administered as indicated in the experimental design of FIG. 12.

FIG. 15 shows the distribution and average value of diarrhea score observed on day 2 (FIG. 15A) and on day 12 (FIG. 15B) of mice treated according to the experimental design of FIG. 12. There are significant differences in diarrhea scores on day 12 between the mice treated with vancomycin 50 mg/kg and nystatin 8,500 IU/kg and those treated with nystatin 8,500 IU/kg. Results are shown as mean±SEM (Standard Error of the Mean). Asterisk indicates P<0.05.

FIG. 16 shows the CFU load of spores in stools of mice that were infected with C. difficile spores and treated according to the experimental design of FIG. 12, every day of the trial. Dotted line indicates limit of detection. Indicated are untreated mice (white triangles), mice treated with nystatin 8,500 IU/kg (diamonds), mice treated with vancomycin 50 mg/kg plus nystatin 8,500 IU/kg (white squares) and mice treated with vancomycin 50 mg/kg (white circles).

FIG. 17 shows the histological damage variables in cecum tissue of mice treated with vancomycin 50 mg/kg, and of mice treated with vancomycin 50 mg/kg together with nystatin 8,500 IU/kg orally according to the scheme of FIG. 12. Shown are distribution and average value of histological scores of cecum samples for: cellular infiltration, FIG. 17A; edema, FIG. 17B; and epithelial damage, FIG. 17C. Results are shown as mean±SEM (Standard Error of the Mean).

FIG. 18 shows the histological damage variables in colon tissue of mice treated with vancomycin 50 mg/kg, and of mice treated with vancomycin 50 mg/kg together with nystatin 8,500 IU/kg orally according to the scheme of FIG. 12. Shown are distribution and average value of histological scores of colon samples for: cellular infiltration, FIG. 18 A; edema, FIG. 18B; and epithelial damage, FIG. 18C. Results are shown as mean±SEM (Standard Error of the Mean).

FIG. 19 shows the distribution and average value of CFU load of C. difficile spores in proximal colon tissue in mice infected with C. difficile spores and treated according to the experimental design of FIG. 12. Tissues were extracted 4 days after finishing the treatment with vancomycin, the time of manifestation of the symptoms of R-CDI. Results are shown as mean±SEM (Standard Error of the Mean).

FIG. 20 shows the distribution and average value of cytotoxicity of the cecal content on Vero cells during the period of recurrence of R-CDI in mice infected with C. difficile spores and treated with vancomycin 50 mg/kg, or vancomycin 50 mg/kg with nystatin 8,500 IU/kg as indicated in FIG. 12. Results are shown as mean±SEM (Standard Error of the Mean).

FIG. 21 shows a scheme of experimental C. difficile infection design using a pharmacological formulation of nystatin and vancomycin to evaluate its use as treatment for CDI and prevention of R-CDI. For this purpose, all mice were treated with a mixture of antibiotics during 3 days, after 2 days they were treated with clindamycin, on the following day they were infected with 1×10⁷ C. difficile spores, they were evaluated for 2 days until manifestation of the initial infection and then were treated with i) vancomycin (n=4), ii) vancomycin 50 mg/kg and nystatin 4,250 IU/kg (n=5), iii) vancomycin 50 mg/kg and nystatin 17,000 IU/kg (n=5) during 5 days. After finishing the treatment with vancomycin, mice were evaluated on a daily basis until day 16 when they were sacrificed.

FIG. 22 shows the average weight of the mice of the experiment indicated in FIG. 21. Shown is the weight in the R-CDI period (after discontinuing treatment). Indicated are mice of the groups treated with i) vancomycin, ii) vancomycin and nystatin 4,250 IU/kg and iii) vancomycin and nystatin 17,000 IU/kg. The group treated with vancomycin had a statistically significant fall in weight in comparison with the mice treated with vancomycin and nystatin. Asterisk indicates P<0.05.

FIG. 23 indicates the time taken by mice infected by C. difficile and treated with i) vancomycin, ii) vancomycin and nystatin 4,250 IU/kg and iii) vancomycin and nystatin 17,000 IU/kg, according to the experimental design of FIG. 21, to present diarrhea associated to R-CDI after finishing administration of the treatment.

FIG. 24 shows the CFU load of C. difficile spores in stools that were collected daily. The dotted line indicates the limit of detection of the technique. Indicated are mice treated with i) vancomycin, ii) vancomycin and nystatin 4,250 IU/kg and iii) vancomycin and nystatin 17,000 UI/kg, according to the experimental design of FIG. 21. No significant differences were observed between the treatments.

FIG. 25 shows a scheme of experimental C. difficile infection design using a formulation based on nystatin, vancomycin and taurocholate, to evaluate the use thereof as a treatment for CDI and prevention of R-CDI.

FIG. 26 shows the average weight of the mice of the experiment indicated in FIG. 25.

FIG. 27 shows the time taken by mice infected by C. difficile and treated with i) vancomycin, ii) vancomycin and nystatin 8,500 IU/kg and iii) vancomycin 50 mg/kg, nystatin 8,500 IU/kg and sodium taurocholate 20 mg/kg (according to the experimental design of FIG. 25), to present diarrhea associated to a recurrent C. difficile infection.

FIG. 28 shows the time taken by mice infected by C. difficile, which manifested severe symptoms of CDI and which were then treated with i) vancomycin and nystatin or ii) vancomycin+ramoplanin+nystatin (following the timeline of the experimental design of FIG. 21), to present diarrhea associated to a recurrent C. difficile infection.

### DETAILED DESCRIPTION OF THE INVENTION

In one embodiment, the present invention refers to nystatin as a drug useful for reducing endocytosis of C. difficile spores and thus reducing diarrhea caused by R-CDI.

In another embodiment, the present invention refers to a composition comprising nystatin and vancomycin, the composition being useful for reducing diarrhea caused by R-CDI.

In another embodiment, the present invention refers to a composition comprising nystatin, vancomycin and taurocholate, the composition being useful for reducing diarrhea caused by R-CDI.

In another embodiment, the present invention refers to a composition comprising nystatin, vancomycin and ramoplanin, the composition being useful for reducing diarrhea caused by R-CDI.

The present invention describes that nystatin reduces endocytosis of C. difficile spores in vitro. Based on these experimental data, the effect of nystatin on R-CDI cases was tested in vivo. For this purpose, mice were infected with spores of C. difficile strain R20291, and were treated with nystatin administered intraperitoneally and orally.

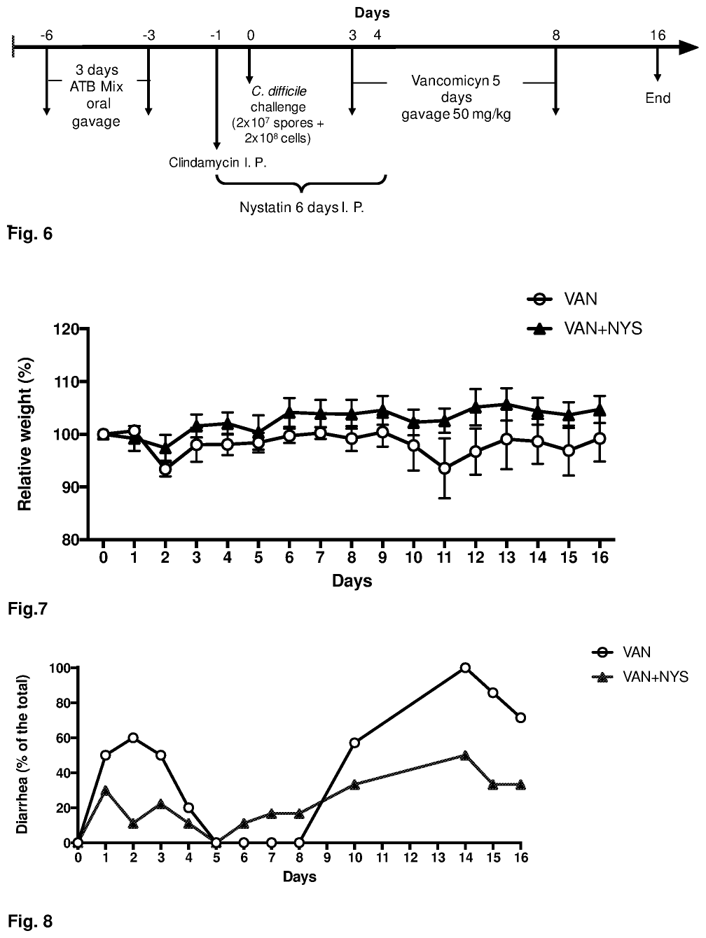

Initially, we assessed the effect of intraperitoneally administered nystatin according to the experimental design indicated in FIG. 6, evaluating the parameters of weight loss, amount of spores in stools and diarrhea.

No significant weight variations were observed between the group of mice treated with nystatin and the group of mice not treated with nystatin (control). Interestingly, mice treated with intraperitoneally administered nystatin tend to a slight weight increase.

Spore abundance in stools begins to increase on the day following the infection, both for the group treated with nystatin and for the control group. As from the vancomycin administration on day 3, the amount of C. difficile colony forming units (CFU) decreases on day 4 down to the limit of detection of the technique used.

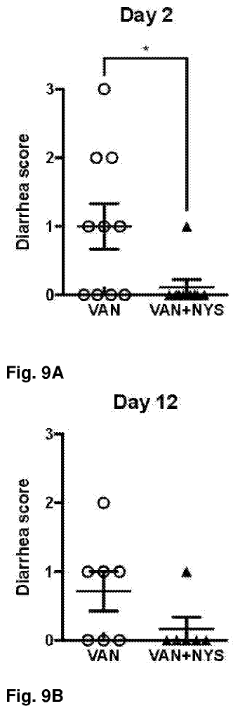

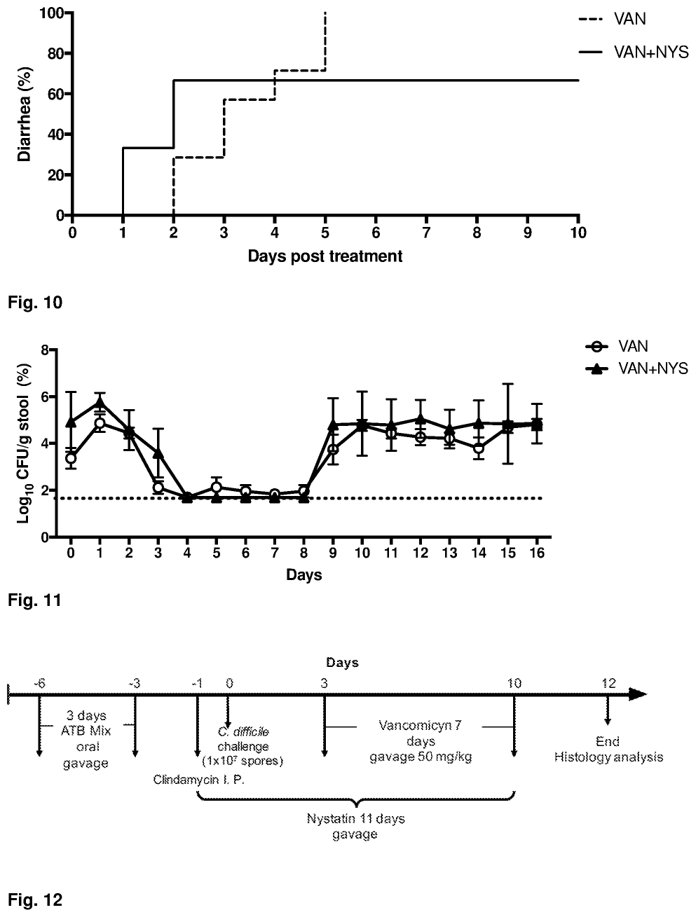

It was observed that 100% of the control mice (not treated with nystatin) exhibited recurrence 5 days after finishing the treatment with vancomycin (FIG. 10). 33% of the mice treated with a composition comprising nystatin and vancomycin did not present recurrence until 10 days after finishing the treatment (FIG. 10). Administration of intraperitoneal nystatin together with oral vancomycin does not reduce the spores eliminated in stools, nor the histological damage. However, treatment with nystatin and vancomycin reduces diarrhea in 67% of the mice treated (FIG. 8).

Additionally, the present invention assessed the administration of a pharmacological formulation of nystatin and vancomycin at the onset of symptoms of C. difficile infection, and it was observed that the administration of a pharmacological formulation of nystatin and vancomycin reduces diarrhea cases caused by recurrent C. difficile infection.

In the present invention, it was observed that the administration of a nystatin formulation together with an antimicrobial agent prior to infection reduces the symptoms of recurrent infections produced by C. difficile. Additionally, it was observed that the administration of a nystatin formulation together with an antimicrobial agent after manifestation of the C. difficile infection is capable of reducing recurrent C. difficile infections.

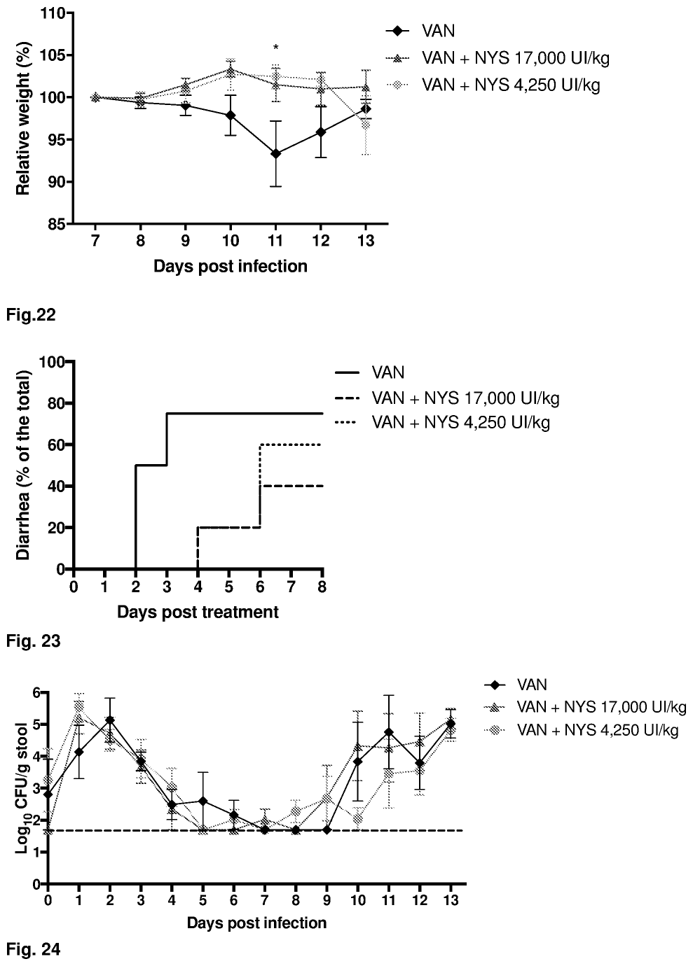

In this sense, it was observed that when beginning to administer a formulation of vancomycin and nystatin at concentrations of 4,250 IU/kg and 17,000 IU/kg, there is an immediate improvement in the weight loss of infected mice (FIG. 22). Likewise, in mice treated with a formulation comprising vancomycin and nystatin at concentrations of 4,250 IU/kg and 17,000 IU/kg, diarrhea manifested itself two days later and affected a lower percentage of mice in comparison with the treatment with vancomycin (FIG. 23). The treatment with a formulation comprising vancomycin and nystatin (17,000 IU/kg) reduced by half the percentage of mice with diarrhea.

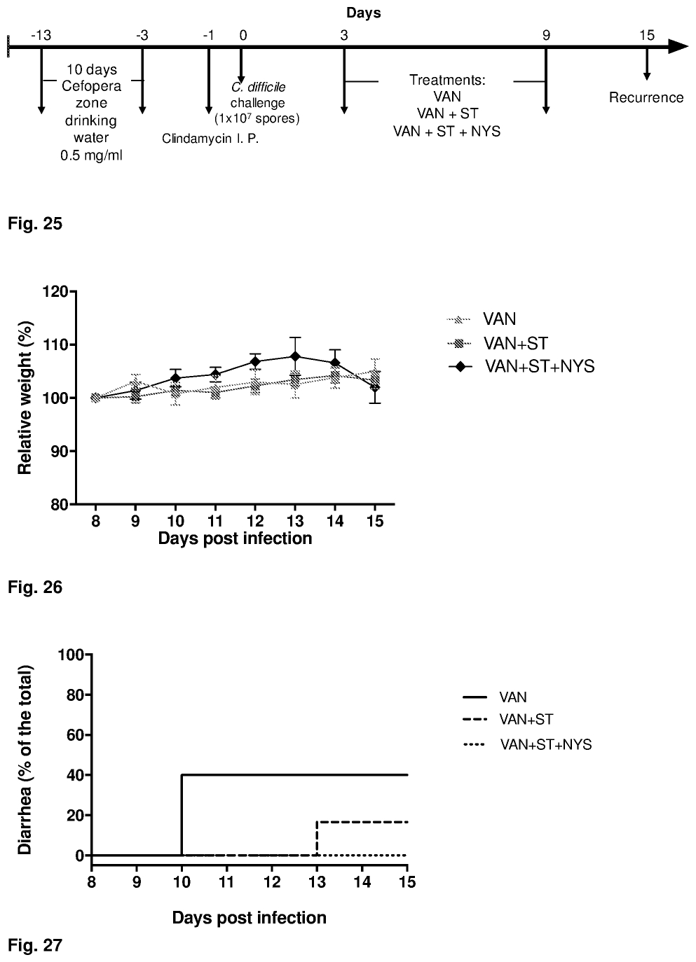

On the other side, we assessed the effect of a formulation comprising vancomycin together with nystatin and a germinant of C. difficile spores (taurocholate), administered orally according to the experimental design indicated in FIG. 25, evaluating the parameters of weight loss and time before manifestation of diarrhea in R-CDI. No significant weight loss was observed in the recurrence for any of the treatments. However, 40% of the mice treated with i) vancomycin exhibited recurrence. Only 20% of the mice treated with ii) vancomycin plus nystatin exhibited recurrence after C. difficile infection. None (0%) of the mice treated with iii) vancomycin, nystatin and taurocholate exhibited recurrence. The results are surprising because they indicate that nystatin reduces by half the incidence of R-CDI and taurocholate increases the activity of the formulation of vancomycin and nystatin in the treatment of R-CDI. Results show that 100% control of R-CDI was achieved (FIG. 27).

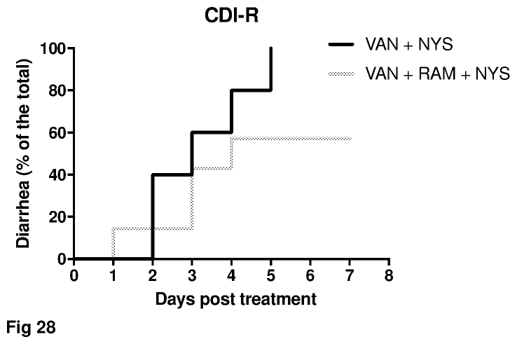

Finally, we studied the effect of a formulation comprising nystatin together with vancomycin and a ramoplanin, the latter administered orally following the timeline of the experimental design of FIG. 21, and evaluated the parameter of time before manifestation of diarrhea in R-CDI. For the purpose of this evaluation, we only considered the mice that manifested diarrhea during the CDI, and consequently, a significant decrease was observed in the time in which the mice manifested diarrhea. However, 100% of the mice treated with vancomycin and nystatin which presented a severe CDI, presented diarrhea, while 57% of the mice treated with vancomycin, ramoplanin and nystatin presented diarrhea. Results show that this formulation achieved 43% control of R-CDI (FIG. 28).

### APPLICATION EXAMPLES

### Example 1

### Treatment of Samples for Analysis by Transmission Electron Microscopy.

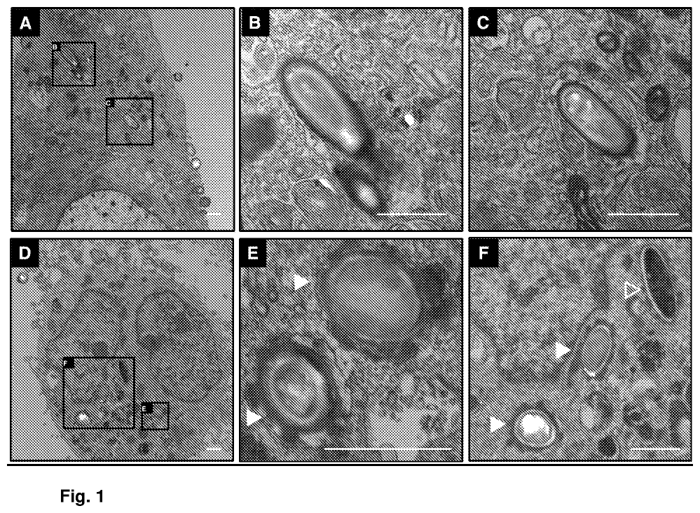

To demonstrate that C. difficile spores are capable of entering differentiated intestinal epithelial cells (IEC), Caco-2 cells were differentiated by culturing them in confluent monolayers for 8 days, and T84 cells were cultured in Transwell (Corning) up to a resistance of 1000-2000Ω (˜14 days post-confluence), and they were infected during 5 hours at MOI 20 with C. difficile spores pre-incubated with normal human serum. They were then washed to remove non-adhered spores, fixed overnight at 4° C. with a 3% glutaraldehyde solution with cacodylate buffer (pH 7.2) and stained during 30 minutes with 1% tannic acid. Subsequently, the samples were processed and dehydrated for 30 minutes in a gradient of 30%, 50%, 70%, 90% and 2 times 100% acetone, containing 2% uranyl acetate, for 30 min each. Dehydrated samples were embedded in resin at an acetone:resin ratio of 3:1, 1:1 and 1:3 for 30 min each, and embedded in spurn resin for 4 hours and then incubated for 12 h at 65° C. Thin 90-nm sections were obtained with a microtome and placed on a carbon-coated grid for negative staining and double staining with 2% uranyl acetate and lead citrate. The sections were observed under the Phillip Tecnai 12 bioTWEIN Transmission Electron Microscope. Electron microscopy images are shown in FIG. 1.

FIG. 1 shows transmission electron micrographs of polarized T84 (FIG. 1A) and Caco-2 (FIG. 1D) cells, which were infected during 5 hours with vegetative cells and spores of C. difficile strains 630 and R20291, respectively, previously incubated with NHS. Cells were washed and processed to be analyzed by transmission electron microscopy. FIGS. 1B and 1C correspond to enlargements of FIG. 1A, where intracellular spores can be observed. FIGS. 1E and 1F correspond to enlargements of FIG. 1D. Scale bars represent 1 μm. It can be observed that C. difficile spores are capable of entering intestinal epithelial cells.

Ultrastructure of the Interaction of C. difficile Spores with Intestinal Epithelial Cells.

By means of transmission electron microscopy (TEM), we observed that C. difficile spores of historical strain 630 are capable of entering T84 cells (FIGS. 1A, 1B, 1C). Additionally, we observed that C. difficile spores of the hypervirulent strain R20291 are capable of entering Caco-2 cells (FIG. 1D). Micrographs show that more than one spore is capable of entering a same cell (FIG. 1A) and the spores that enter the cell are inside a vesicle closely associated thereto (FIGS. 1B, 1C). Due to the difficulty we had in finding this phenomenon in both cell lines, we might presume that this phenomenon occurs with low frequency within a cell population.

### Example 2

Internalization of C. difficile Spores In Vivo.

Based on the results observed for the entry of C. difficile spores into intestinal cells in monolayers, we have studied whether C. difficile spores are capable of entering mouse intestinal cells.

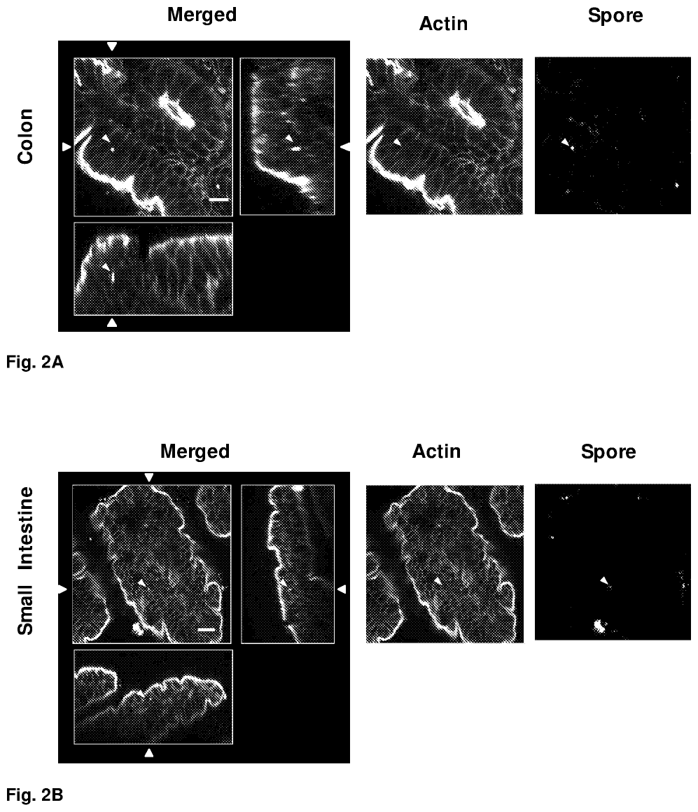

For the in vivo study, we used 6- to 12-week old mice which were infected in the ileum of the small intestine and in the ascending zone of the large intestine by the intestinal obstruction technique for a period of 6 hours, and they were processed to be analyzed by confocal microscopy. It was observed that spores adhere to a greater extent to the small intestine than to the large intestine, and that spores adhere to the lumen of the intestinal tissue. Additionally, it can be observed that spores can enter and distribute themselves in the crypts of the small intestine, referred to as crypts of Lieberkühn (FIG. 2B). Only some spores are capable of entering crypt cells (see the spores that are indicated with an arrow head in FIGS. 2A and 2B). This study demonstrates that C. difficile spores can enter intestinal epithelial cells, and this could be a mechanism for promoting the persistence of spores in the intestine and for causing recurrent C. difficile infections.

For this study, 6- to 12-week old mice were fasted overnight, and anesthetized with 100 mg/kg of ketamine and 10 mg/kg of xylazine. A small incision was made in the abdominal wall to expose the cecum. Then, the obstruction of the ileum (final portion of the small intestine) and of the ascending large intestine (initial portion of the large intestine) was performed, and 2.5×10⁸ spores per cm² were injected into the obstructed zone. Intestines were returned to the abdominal cavity, suturing with silk thread (surgery performed under surgical sterile conditions). Once the mice recovered from anesthesia, they were maintained for 6 hours after surgery in front of an infrared light heat source. After the 6 hours, the mice were anesthetized with the above described dose, the obstructed sections were extracted by cutting the ends of the knots, and the tissues were cleaned and washed under a biosafety hood. Tissues were fixed overnight with 4% paraformaldehyde with 30% sucrose. Tissues were then washed and a 5×5-mm segment was directly stained and mounted. Tissues were permeabilized with 0.2% Triton X-100 in phosphate buffered saline (PBS) for 2 hours, washed with PBS and blocked with 3% bovine serum albumin (BSA) in PBS for 3 hours. Tissues were incubated with 1:1000 anti-spore IgY antibodies overnight. They were then washed and incubated with 1:300 568 IgY antibodies for 3 hours. They were washed twice and incubated with 1:50 phalloidin 488 overnight. Samples were washed and labeled with Hoechst 2 mg/kg for 30 minutes. Tissues were then mounted and photographed by a Leica TCS LSI confocal microscope, using a 63× lens. Images were processed with ImageJ. FIG. 2A shows an image of the colon. FIG. 2B shows an image of the small intestine. Images are representative of 3 different sites, analyzed in 3 different mice. Arrows indicate spores found within the tissue. Scale bar represents 10 μm. FIG. 2 shows that C. difficile spores are capable of entering mouse cells of the small intestine and the large intestine.

FIG. 2 shows confocal microscopy images of 1-cm² sections of small intestine and large intestine of C57BL/6 mice that were infected with 2.5×10⁸ C. difficile spores per cm² during 6 hours by the ileal loop technique. Then, the infected intestine sections were extracted, fixed with 4% PFA with 30% sucrose overnight and processed for direct immunofluorescence. Tissues were blocked, the actin cytoskeleton was labeled with phalloidin (green), and the spores were stained with polyclonal IgY antibodies against C. difficile spores and detected with secondary antibody (red). Finally, they were observed by confocal microscopy.

### Example 3

### Internalization of Spores is Reduced by Nystatin In Vitro.

Since C. difficile spores are endocytosed by intestinal epithelial cells and by Lieberkühn crypt cells, it is conceivable to suppose that this would be one of the mechanisms by which spores could persist in the host to cause recurrent infections. Therefore, we conducted in vitro studies searching for drugs capable of reducing spore entry. The drugs evaluated were nystatin, chlorpromazine and indomethacin.

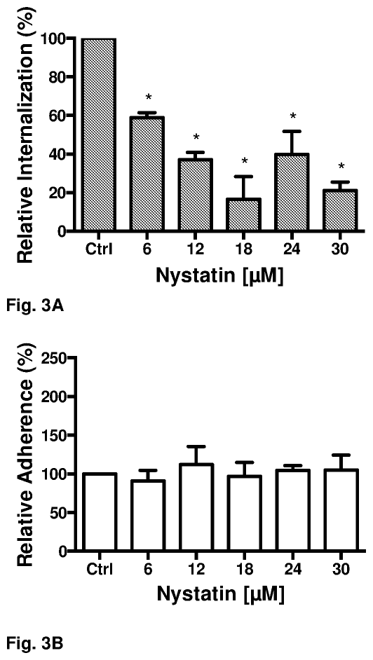

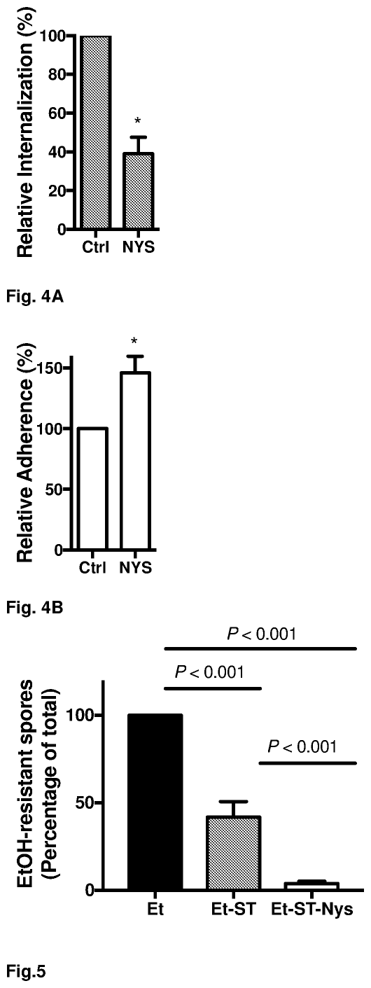

To evaluate spore internalization, Caco-2 and T84 cells were seeded onto 24-well plates on a glass coverslip up to 2 days after confluence. The cells were then preincubated during one hour with 6, 12, 18, 24, 30 μM nystatin; 20, 40, 60, 80 and 100 μM chlorpromazine and 50, 100, 150, 200 and 250 μM indomethacin in Dulbecco's Modified Eagle's medium (DMEM) high in glucose, 1% penicillin and streptomycin. At the same time, spores of C. difficile strain R20291 were incubated at 37° C. with FBS. Then the cells were infected with the spores at MOI 10, in a 10% FBS solution for 3 hours in the presence of nystatin. Cells treated with the solvent of the inhibitor (Ctrl) and 10% FBS were used as control. The wells were then washed twice with Dulbecco's Phosphate-Buffered Saline (DPBS) to remove non-adhered spores. Subsequently, to determine whether C. difficile spores are inside the cells, the previously fixed cell monolayers were blocked overnight with 1% FBS at 4° C. and extracellular spores were labeled with anti-spore goat serum diluted 1:50 in 1% FBS-DPBS for one hour at room temperature. Labeled spores were washed twice with DPBS and incubated with 1:400-diluted CruzFluor™ 488 (CFL 488) conjugated bovine anti-goat IgG (green) (Santa Cruz Biotechnology), washed 3 times with DPBS and then submitted to a final wash with distilled water to remove excess salts. Samples were dried at 37° C. for 30 min, mounted with Dako fluorescence mounting medium (Dako) and sealed with nail polish. Samples were analyzed with an Olympus BX53 epifluorescence microscope. C. difficile spores that had entered the cell were analyzed one by one as spores visible in phase contrast and not visible by green fluorescence, while the extracellular or adhered spores were identified as spores visible in phase contrast that were labeled with green fluorescence; and it was observed that all three drugs are capable of reducing spore internalization in vitro (data not shown). However, the major reduction in internalization was observed with nystatin (FIG. 3A), and we saw that nystatin significantly reduces, in a process dependent on the inhibitor concentration, internalization of spores by Caco-2 cells, exhibiting a reduction of spore entry by up to 79% in respect of the control. In T84 cells, nystatin reduced spore entry by 60% in respect of the control (FIG. 4A). In Caco-2 and T84 cells, no differences were observed in cell viability by the trypan blue and MTT methods (results not shown). In Caco-2 cells adherence was not affected (FIG. 3B), however in T84 adherence increased by 46% (FIG. 4B), suggesting that in this cell line, there are compensation mechanisms to the use of this drug, which are employed by the spores for their adherence. It should be noted that nystatin is approved as a drug by the FDA and the side effects from the use of nystatin are minimal. For this reason, it was selected to continue with the in vivo studies.

FIG. 3A shows that internalization of C. difficile spores in intestinal epithelial cells is inhibited by nystatin. Asterisks in FIG. 3A indicate P<0.05.

FIG. 3B shows that spore adherence is not interfered with by nystatin in Caco-2 cells. In FIG. 3B, no significant differences were observed.

The same experiment was conducted in T84 cells. Cells were treated with increasing nystatin concentrations for 1 hour and then infected during 3 hours in the presence of nystatin with MOI 10 of R20291 spores, pre-incubated for 1 hour with FBS. T84 cells were infected with the highest concentration used in Caco-2. They were then fixed and treated to be analyzed by epifluorescence microscopy. Values were compared by t-test in respect of the control.

Results of internalization and adherence of spores in T84 cells, in the presence of different nystatin concentrations, are shown in FIG. 4A and FIG. 4B.

On the other side, the capacity of nystatin for reducing the amount of internalized spores was evaluated by the seeding method.

For this purpose, Caco-2 cells at 2 days of confluence were treated with 30 μM nystatin or with its carrier (DMSO) during one hour. Cells were then infected with C. difficile R20291 spores at MOI 10 for a period of 3 h. Considering that endocytosed spores are protected from the bile salts of the colonic lumen, contrary to adhered spores, cells were washed and incubated with 0.1% taurocholate in DMEM with 10% FBS and were incubated at 37° C. for 1 h to germinate the C. difficile spores having access to taurocholate. Subsequently, since spores are by nature persistent to ethanol, as opposed to vegetative cells, the infected monolayers were washed and incubated with 70% ethanol for 15 min. The cells were then lysed with a 0.2% triton solution and seeded onto BHIS plates supplemented with 0.1% taurocholate to germinate the spores that had resisted the initial treatment, that is, the endocytosed spores. Plates were incubated for 2 days in anaerobiosis.

After concluding the analysis, we can see that treatment with taurocholate (ST) for a period of 1 h and then with ethanol, inactivates approximately 50% of the total spores (FIG. 5), which by nature are persistent to ethanol. However, in cells incubated with nystatin before and during the infection, a significant decrease in the amount of CFU counted was observed, suggesting that nystatin blocks entry of spores into IEC, which allows the treatment with taurocholate and then with ethanol to eliminate most of the persistent spores.

Results of percentage of CFU of spores resistant to ethanol in Caco-2 cells infected with C. difficile spores pretreated with nystatin are shown in FIG. 5.

### Example 4

### Administration of I.P. Nystatin Before and During a CDI is Capable of Reducing Diarrhea Caused by R-CDI.

Based on the studies of example 3, in which we found a drug such as nystatin that reduces spore endocytosis in vitro, we wondered whether the use of this drug reduces the R-CDI cases in vivo. For this purpose, we infected C57BL/6 mice with spores of C. difficile strain R20291 and treated the mice with nystatin administered intraperitoneally and orally.

We initially evaluated the effect of intraperitoneally administered nystatin, according to the experimental design indicated in FIG. 6, and evaluated the parameters of weight loss, amount of spores in stools and diarrhea.

The 8 mg/kg nystatin solution was prepared in intralipids by dissolving nystatin (25 mg/ml in DMSO) at a concentration of 1 mg/ml in a 20% solution of intralipids, and mixing for 24 h with stirring at 280 rpm.

We observed that there are no significant weight variations between the group treated with nystatin and the control group. Interestingly, mice treated with intraperitoneally administered nystatin tend to a slight increase in weight, reaching an increase of 6% on day 11 in respect of day 0 (FIG. 7).

While spore abundance in stools starts to increase on the day following infection (day 1), both for the group treated with nystatin and for the control group, since the vancomycin administration on day 3, the amount of CFU decreases on day 4 down to the limit of detection of the technique used. Once the treatment with vancomycin is discontinued on day 9, an increase in CFU is observed on day 10, up to levels comparable with the amount of spores in stools in the initial condition (FIG. 11).

Interestingly regarding diarrhea in the initial condition, observed on day 2, 60% ( 6/10) of the control mice presented diarrhea, while 11% ( 1/9) of the mice treated with vancomycin+nystatin presented diarrhea (FIG. 8). On day 7, after finishing treatment with vancomycin, 3 mice were randomly chosen to evaluate histology, and in the variables of damage, edema and cellular infiltration, no significant differences were observed between both groups (results not shown).

Mice were monitored up to day 16 (9 days after vancomycin) and we observed that 100% of the control mice presented recurrence 5 days after finishing treatment with vancomycin, while 33% of the treated mice did not present recurrence (FIG. 10). In summary, administration of intraperitoneal nystatin together with oral vancomycin does not reduce the spores eliminated in stools nor the histological damage, but reduces diarrhea in 33% of the treated mice.

We conducted a study on a murine model in which C57BL/6 mice were infected and dosed with oral antibiotic and intraperitoneal nystatin, according to the scheme of FIG. 6.

For this study, wild C57BL/6 mice, 8-9 weeks old, were housed in previously sterilized individual cages, placed under 12-hour light/darkness cycles and maintained with miliQ water, food and sterile wood shavings. To generate a dysbiosis of intestinal microflora and make the mice prone to a CDI, they were administered an antimicrobial treatment by the oral orogastric route for 3 days. The antimicrobial treatment consists of a solution composed of kanamycin 40 mg/kg, gentamycin 3.5 mg/kg, colistin 4.2 mg/kg, metronidazole 12.5 mg/kg and vancomycin 4.5 mg/kg in a 100-μl volume of saline. They were then provided with sterile miliQ water without antimicrobials. One day prior to infection, the mice were dosed with 30 mg/kg of intraperitoneal clindamycin. On the day of infection, the weight of the mice was assessed and a stool sample was collected to verify that none was infected by C. difficile. Subsequently, they were infected orally with 1×10⁷ C. difficile R20291 spores.

We evaluated intraperitoneal administration of nystatin (12,000 IU/kg in intralipids), which was given from day −1 until the last day of vancomycin administration with the intention of reducing spore endocytosis. Vancomycin was administered for 7 days at a dose of 50 mg/kg to kill C. difficile vegetative cells. The mice were observed until the group not treated with nystatin presented symptoms of R-CDI.

We observed that intraperitoneal nystatin administration reduces diarrhea in R-CDI.

To evaluate the effect of intraperitoneally administered nystatin on R-CDI, a murine model of recurrence of R-CDI was used, as described by Sun et al. (2011), with the modifications described herein.

FIG. 6 shows a scheme of experimental infection design. All mice were treated with a mixture of 5 antibiotics for 3 days, then they were evaluated during 2 days and the next day they were treated with clindamycin; 9 mice were treated with nystatin and 10 mice were not treated with nystatin (control). On the following day, they were infected with 1×10⁷ C. difficile spores and were evaluated during 2 days when the initial infection manifests, and then they were treated with vancomycin for 7 days. After finishing the treatment with vancomycin, 3 mice were extracted for histological analyses of cecum and colon. Mice were evaluated daily until day 16 when they were sacrificed.

### Example 5

### Oral Administration of Nystatin Before and During a CDI Reduces Diarrhea Caused by R-CDI.

We then wondered whether oral administration of nystatin was more effective than intraperitoneal administration, for which purpose an oral nystatin composition was prepared in a 40% v/v ethanol solution in distilled water.

We evaluated the oral administration of nystatin (8,500 IU/kg) which was given from day −1 until the last day of vancomycin administration with the intention of reducing spore endocytosis. However, the period of vancomycin administration was different. Vancomycin was administered for 5 days at a dose of 50 mg/kg to kill C. difficile vegetative cells. The mice were observed daily until the group not treated with nystatin presented symptoms of R-CDI.

We observed the effect of orally administered nystatin on R-CDI. For this purpose, we used the murine model of recurrence of CDI as described in Example 4. FIG. 12 shows a scheme of experimental infection design.

To conduct this study on oral administration of nystatin, we evaluated 5 mice treated only with vancomycin, 5 with vancomycin and nystatin, and as controls, 4 untreated infected mice and 4 mice treated only with nystatin. In this case, we reduced the time of use of vancomycin from 7 to 5 days as shown in FIG. 12, similar to the days of administration in humans.

In this trial, we evaluated the variables of weight loss (FIG. 13), CFU in stools (FIG. 16), diarrhea, histological damage on the day of recurrence (day 12), amount of spores adhered to colon tissue and, as an indicator of C. difficile toxigenic culture, toxin levels in cecal content.

No significant differences were observed in weight variation between the group treated with vancomycin and the group treated with vancomycin+nystatin. However, untreated infected mice tend to a 10% weight increase at the end of the experiment, relative to the initial day (FIG. 13).

The treatment with vancomycin+nystatin showed the lowest levels of spores in stools on day 2 (3.0 logarithmic units) in comparison with mice treated only with vancomycin (3.8 logarithmic units). However, on day 3 these values reverse, while on day 11 (the day before recurrence) similar levels of spores in stools are observed in all mice, except for the mice treated only with nystatin which exhibit elevated values for CFU in stools (FIG. 16).

### Weight Measurement and Determination of Diarrhea Score

Mice were observed daily and their weight was measured and compared with the weight on the day of infection. Diarrhea was evaluated visually according to a diarrhea score, where 0 is for normal stools, 1 when softening and/or color change (yellow) is observed in respect of day 1, 2 when the mouse tail appears wet and/or there is mucus in the stool, and 3 when the stool is liquid or there is absence of stool.

FIG. 13 shows the average daily weight of mice treated with vancomycin as control (white circles) and treated with vancomycin plus nystatin (white squares).

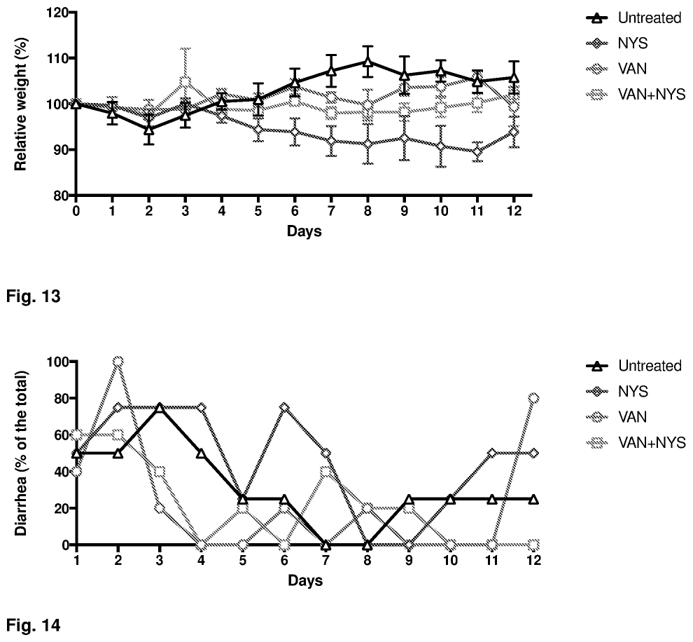

FIG. 14 shows the average percentage of mice with diarrhea, in mice treated with vancomycin and nystatin, diarrhea decreases by 40% on day 2 and by 80% on day 12.

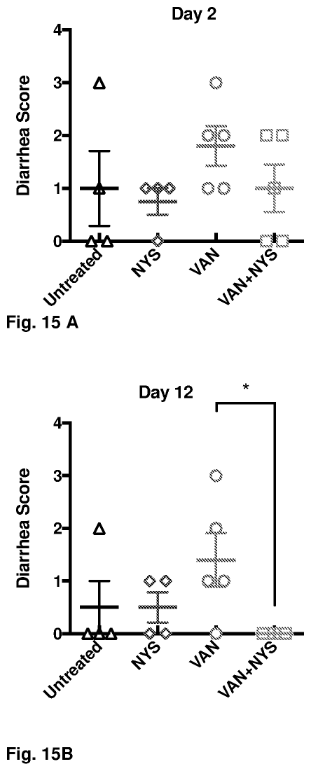

FIGS. 15A and 15B show the diarrhea scores observed on days 2 and 12, respectively. Asterisks indicate P<0.05.

Interestingly, FIGS. 14 and 15 show that on day 12, 25% (¼) of the untreated mice presented diarrhea, 50% ( 2/4) of the mice treated with nystatin presented diarrhea, 80% of the mice treated with vancomycin presented diarrhea and none (0%) of the mice treated with vancomycin+nystatin presented diarrhea (FIGS. 14 and 15). This indicates that the administration of a formulation comprising vancomycin+nystatin protects mice from recurrence of C. difficile infection.

### Determination of Colony Forming Units (CFU) in Stools

To determine the CFU load of spores in stools, the stools were hydrated, treated with ethanol, macerated and seeded onto TCCFA plates. Dotted line indicates limit of detection.

For this study, the stool samples collected were hydrated with 500 μl of PBS overnight at 4° C. The next day the samples were homogenized, ethanol-resistant organisms (such as spores) were selected by adding 500 μl of 100% ethanol for 20 minutes, the samples were then serially diluted and seeded onto plates with 4% protease peptone, 0.6% fructose, 0.1% Na₂HPO₄, 0.1% KH₂PO₄, 0.2% NaCl, 0.02% MgSO₄, 250 μg/ml cycloserine, 15 mg/ml cefoxitin supplemented with 0.1% sodium taurocholate (TCCFA), this being a selective medium for C. difficile which allows for its germination and for CFU counting.

FIG. 16 shows the CFU load of spores in stools of mice that were infected with C. difficile spores and treated according to the experimental design of FIG. 12. In FIG. 16, the dotted line indicates limit of detection.

### Determination of Histological Damage in Cecum and Colon Samples

Cecum and colon tissues were extracted from the group of mice treated with oral nystatin together with vancomycin and from the group of mice treated with vancomycin, on day 12, as indicated in Example 5, to be analyzed in respect of the variables of histological damage, and it was determined that nystatin does not reduce damage to the intestinal epithelium during R-CDI.

Based on the fact that we were able to reduce the diarrhea cases in CDI and R-CDI but the spore load was not reduced, and since the spore load does not have a pattern during the treatment, we wondered whether nystatin is capable of reducing the damage caused by the infection at the histological level, both in cecum and colon (FIGS. 17 and 18). Based on the scores for histological damages defined as cellular infiltration, edema and epithelial damage, no differences were observed between both groups, indicating that nystatin is not capable of reducing tissue damage, or else, given the absence of diarrhea on day 12, the damage observed may have been caused during the initial infection and has not healed by the end of the experiment.

Once the cells have been fixed, the tissue is washed 3 times with 1 ml of sterile PBS. For embedding tissues in paraffin, these were dehydrated in increasing ethanol concentrations, 70, 95 and 100% for 30 minutes each. Tissues were left to stand in Histoclear (National Diagnostics) for 30 min, then incubated in a 1:1 solution of colorless liquid paraffin and Histoclear for 30 min at 56-60° C., subsequently tissues were incubated with paraffin for 2 hours at 56-60° C. in a molding vessel and left to stand until solidification. The blocks were stored at −20° C. until performing the cuts. The blocks were processed with a microtome to achieve cuts of 5 μm thickness. The tissues were then deparaffinized with Histoclear for 5 minutes, and hydrated with decreasing ethanol solutions of 100, 95 and 70% for 5 minutes each. The samples were then dried, hematoxylin (Merck) was added for 2 minutes, they were washed twice with distilled water for 5 minutes and left to dry, and then eosin (Merck) was added during 1 minute. The tissues were dehydrated again with increasing ethanol solutions, 70, 95 and 100% for 5 minutes. They were finally washed with Histoclear for 10 minutes and left to dry. Then the samples were mounted with Histomount mounting solution (National Diagnostics), covered with coverslips and finally sealed with transparent nail polish and analyzed by clear field microscope.

In histological analyses of colon and cecum (the places where C. difficile infection occurs in mice), we observed no differences in the variables of cellular infiltration, edema and epithelial damage (FIGS. 17 and 18).

FIG. 17 shows distribution and average value of the histological scores of cecum samples of mice treated with vancomycin 50 mg/kg, and of mice treated with vancomycin 50 mg/kg together with oral nystatin 8,500 IU/kg according to the scheme of FIG. 12. The evaluated parameters correspond to: cellular infiltration, FIG. 17A; edema, FIG. 17B; and epithelial damage, FIG. 17C. We observed that nystatin does not reduce histological damage in cecum samples during R-CDI in treated mice.

FIG. 18 shows distribution and average value of the histological scores of colon samples of mice treated with vancomycin 50 mg/kg, and of mice treated with vancomycin 50 mg/kg together with oral nystatin 8,500 IU/kg according to the scheme of FIG. 12. The evaluated parameters correspond to cellular infiltration, FIG. 18A; edema, FIG. 18B; and epithelial damage, FIG. 18C. We observed that nystatin does not reduce histological damage in colon samples during R-CDI in treated mice.

We then evaluated other variables of the pathogenesis that could promote recurrence of the infection, such as the amount of spores adhered to the large intestine and the amount of toxins in the cecum, and we observed that treatment with vancomycin+nystatin significantly reduces spore adherence to tissue (P=0.020).

Determination of Spore Abundance in Colon Tissue in Mice Treated with a Pharmacological Formulation Against R-CDI

To evaluate the spore load in the colon, tissues were extracted, then weighed and adjusted to a concentration of 0.1 mg/μl, and mechanically macerated in a Dounce homogenizer. Then the samples were sonicated, serially diluted in PBS and seeded onto TCCFA plates, which were incubated in anaerobiosis for 2 days and CFU were counted.

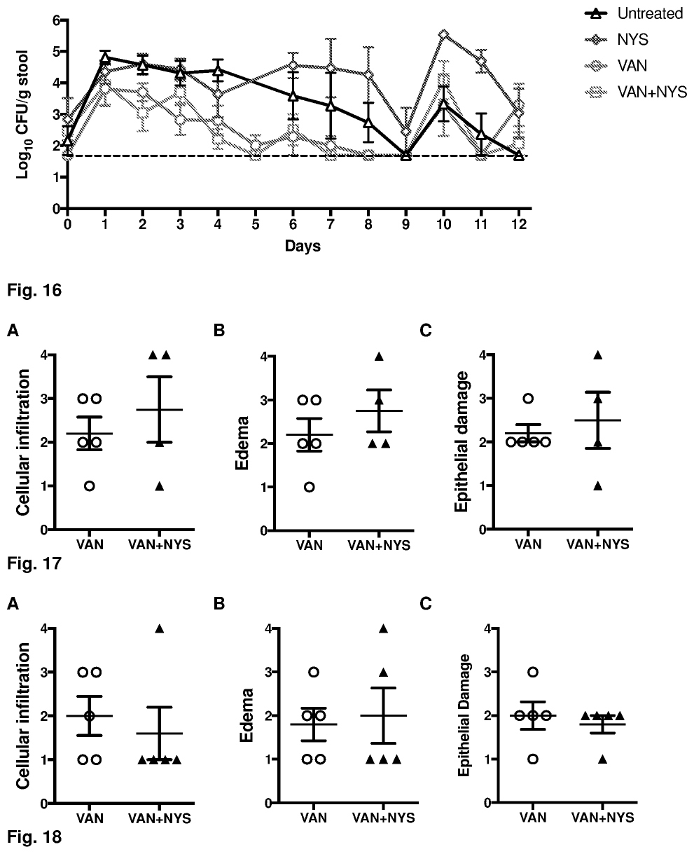

FIG. 19 shows that the formulation of vancomycin and nystatin significantly reduces the amount of spores in colon tissue after the treatment.

### Necropsy and Biological Sample Collection

On the last day of the trial, day 12, mice were anesthetized by intraperitoneal injection with a solution of 40 mg/kg ketamine (Agrovet) and 5 mg/kg xylazine (Centrovet) dissolved in 1×PBS to a final volume of 150 μl. We waited for absence of reflexes of the mouse (˜10 min) before proceeding to sample collection.

Once the mouse was anesthetized, an incision was made in the zone of the abdomen corresponding to the large intestine and both the cecum and the colon were extracted. To evaluate the cytotoxic effects of the cecum luminal content, a sample of same was collected and then both cecum and colon were washed with abundant PBS, and fixed in a 4% paraformaldehyde solution overnight at 4° C.

### Cytotoxicity of the Cecal Content

To determine the cytotoxicity of the cecum luminal content, samples of cecum luminal content were weighed and adjusted to 0.1 mg/μl of PBS, then the samples were homogenized and diluted to 1:10, 1:100 and 1:1000. 100 μl of each dilution were added onto Vero cells seeded in 96-well plates. Antitoxin serum was used as a negative control and purified toxins were used as a positive control. Cells were incubated overnight at 37° C. in 5% CO₂. Vero cells have an elongated shape but when they die, they lose that structure and become rounded. Therefore the following morning, the percentage of rounded cells was determined. The cytotoxicity title was calculated as the reciprocal of the highest dilution which produces the rounding of at least 80% of the cells per gram of cecal content. No significant differences were found between the toxin titles when comparing the treatment with vancomycin alone and the treatment with vancomycin and nystatin.

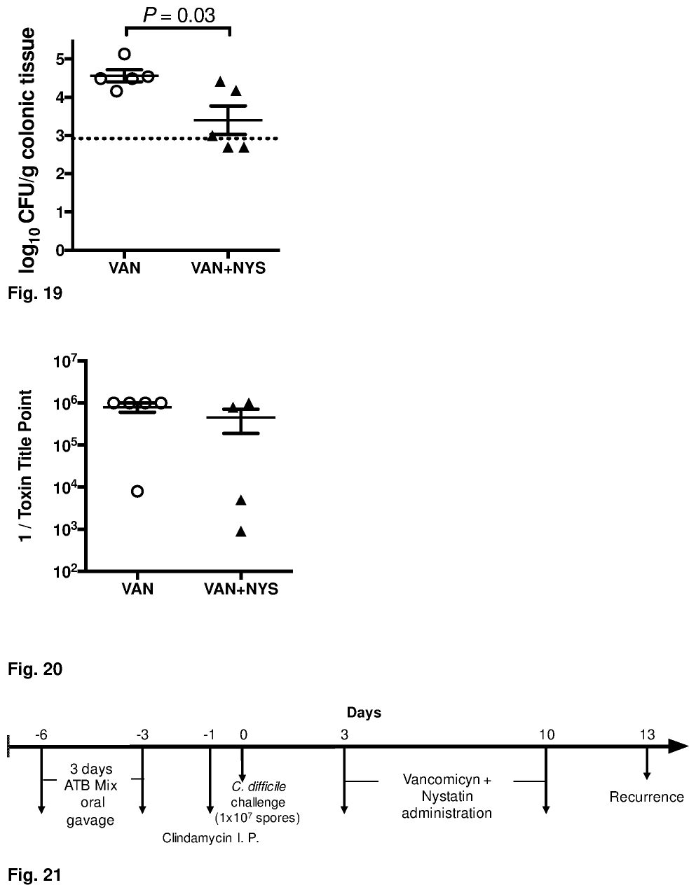

FIG. 20 shows the cytotoxicity of the cecal content on Vero cells in the period of recurrence of R-CDI in mice infected with C. difficile spores and treated with vancomycin 50 mg/kg, or vancomycin 50 mg/kg with nystatin 8,500 IU/kg, as indicated in FIG. 12.

### Nystatin Reduces the Spore Load in Colon Tissue, but not the Cytotoxicity of the Cecal Content.

As the reduction in diarrhea on day 12 by nystatin does not imply less damage to the epithelium, we then wondered whether treatment with nystatin affects spore adherence to colon tissue.

For conducting this study, part of the ileum was macerated and seeded on TCCFA plates and we observed that spores adhered to the colon are significantly reduced in mice treated with nystatin (FIG. 19). It can even be observed that in the colon of 2 mice treated with nystatin, adherence was equivalent to the limit of detection.

Consequently, we wondered whether the cecum content of treated mice has less cytotoxic effects than the control. To evaluate this, Vero cells were challenged with supernatant of the cecal content obtained on day 12 and we observed the loss of their normal shape the following day.

To our surprise, we found that these 2 mice exhibiting no CFU load in the colon, had a diarrhea score of 0. Based on the above, we wondered whether there exists a direct correlation between diarrhea score and CFU load/g of tissue, and we obtained a correlation coefficient (R²) of 0.7, i.e., there exists a positive correlation between both variables. Therefore, it is of interest to increase the amount of biological replicates to establish a trend line, which would allow us to predict the amount of spores adhered to intestinal tissue according to the type of diarrhea the mouse has, which could possibly be extrapolated to humans.

In summary, in mice treated with nystatin, C. difficile infection maintains its course at the lumen level, i.e., growth of vegetative cells, toxin production and spore generation. However, the adherence of these spores to cells of the host is reduced, whereby spore entry into cells of the colon epithelium is reduced and, consequently, the amount of spores in intracellular tissues is reduced. The spores in intracellular tissues bear the greatest responsibility for recurrence of the C. difficile infection.

### Example 6

Treatment with a Formulation Comprising Nystatin and Vancomycin after Onset of Symptoms of C. difficile Infection. Two Formulations, with Different Nystatin Concentrations, were Evaluated.

We studied the effect of the administration of a pharmacological formulation comprising nystatin and vancomycin upon the onset of symptoms of C. difficile infection. We found that the pharmacological formulation of nystatin and vancomycin reduces the cases of diarrhea caused by recurrent C. difficile infection.

From the results of examples 4 and 5, it was observed that nystatin administration prior to infection and during antimicrobial treatment reduces the symptoms of recurrent infections produced by C. difficile.

In view of the foregoing, we desired to find out whether the administration of a formulation comprising nystatin together with an antimicrobial agent after manifestation of the clinical conditions of C. difficile infection is capable of reducing recurrent C. difficile infections.

Infection in C57BL/6 Mice and Treatment with an Oral Formulation Comprising an Antibiotic and Nystatin at Different Doses

Using the same methodology of the recurrence model of C. difficile infection described in example 4, mice were treated with the mix of 5 antibiotics and with the clindamycin dose on the day before infection. Mice were infected and dosed with an oral solution comprising a combination of nystatin and vancomycin starting from day 3 after infection (time of manifestation of the clinical symptoms).

In order to find out the concentration range in which the formulation has activity, two compositions were prepared using as a reference the one used in examples 4 and 5, which corresponds to vancomycin 50 mg/kg together with nystatin at a concentration of 8,500 IU/kg. The compositions chosen as example correspond to half and twice the nystatin dose used in examples 4 and 5, keeping the vancomycin concentration constant, i.e., i) vancomycin 50 mg/kg and nystatin 17,000 IU/kg and ii) vancomycin 50 mg/kg with nystatin 4,250 IU/kg.

FIG. 21 shows a scheme of experimental infection design of the administration of the pharmacological formulation of nystatin and vancomycin for treating R-CDI. The effect of 3 formulations was evaluated; i) vancomycin (n=4), ii) vancomycin+nystatin 4,250 IU/kg (n=5) and iii) vancomycin+nystatin 17,000 IU/kg (n=5).

In this sense, we studied the effect of a formulation comprising nystatin together with vancomycin administered from day 3. 2 nystatin doses, 4,250 IU/kg and 17,000 IU/kg, were evaluated with a same vancomycin dose corresponding to 50 mg/kg dissolved in Dulbecco's phosphate buffered saline solution.

We observed that at the beginning of the administration of vancomycin and nystatin, the weight loss of infected mice improves (not shown). After discontinuing administration of the formulation (day 8), we observed that the mice treated only with vancomycin suffered a weight loss on day 11, the day of recurrence, while the groups treated with a formulation comprising vancomycin and nystatin at concentrations of 4,250 IU/kg and 17,000 IU/kg, did not suffer weight loss (FIG. 22).

Likewise, of the mice treated with vancomycin, 75% presented recurrence, of those treated with vancomycin and nystatin 4,250 IU/kg, 60% presented diarrhea, and with the treatment of vancomycin and nystatin 17,000 IU/kg, only 40% presented diarrhea. Interestingly, it was observed that in both groups treated with vancomycin and nystatin, manifestation of diarrhea was two days later than in the groups treated with vancomycin (FIG. 23).

FIG. 23 indicates the time taken by mice infected by C. difficile and treated with i) vancomycin, ii) vancomycin and nystatin 4,250 IU/kg and iii) vancomycin and nystatin 17,000 IU/kg, according to the experimental design of FIG. 21, to present diarrhea associated to R-CDI after finishing administration of the treatment.

FIG. 24 indicates the CFU abundance of C. difficile spores in stools from mice treated with a formulation comprising i) vancomycin, ii) vancomycin 50 mg/kg and nystatin 4,250 IU/kg and iii) vancomycin 50 mg/kg and nystatin 17,000 IU/kg, according to the experimental design of FIG. 21.

### Example 7

Treatment of CDI with a Formulation Comprising Nystatin, Vancomycin and Taurocholate Reduces the Cases of Diarrhea by R-CDI.

We studied the effect of the administration of a pharmacological formulation comprising nystatin, vancomycin and taurocholate after the onset of symptoms of C. difficile infection. We found that the pharmacological formulation of nystatin, vancomycin and taurocholate reduces the cases of diarrhea caused by R-CDI infection.

From the results of the previous examples, it was observed that a formulation comprising nystatin and vancomycin serves for the treatment of a C. difficile infection, because it reduces the symptoms of recurrent C. difficile infection. However, in order to increase the efficiency of the formulation, a spore germinant was added. The spore germinant has the property of changing the C. difficile morphotype from spore form to vegetative form, the vegetative form of C. difficile being the one susceptible to the effect of an antibiotic, such as vancomycin, which is within the formulation.

In this sense, we administered a formulation comprising nystatin 8,500 IU/kg, vancomycin 50 mg/kg and taurocholate (ST) 20 mg/kg in a 35% v/v ethanol solution, starting from day 3 after the infection with C. difficile as indicated in the scheme of FIG. 25.

We observed that when administering the formulation of vancomycin, nystatin and taurocholate, there were no significant differences in weight in the group of mice treated with the formulation of vancomycin, nystatin and taurocholate (FIG. 26).

Of the mice treated only with vancomycin, 40% presented recurrence, while of those treated with vancomycin and taurocholate, 20% presented diarrhea, and surprisingly, none of the mice treated with a formulation comprising vancomycin, taurocholate and nystatin presented diarrhea (FIG. 27).

FIG. 25 shows a scheme of experimental C. difficile infection design using a formulation based on nystatin, vancomycin and taurocholate, in order to evaluate its use as a treatment for CDI and prevention of R-CDI. For this purpose, all mice were treated with 50 mg/kg of oral cefoperazone during 10 days to generate dysbiosis of intestinal microbiota, three days later they were treated with clindamycin and on the following day, they were challenged with 1×10⁷ C. difficile R20291 spores. They were evaluated for 2 days until manifestation of the initial infection, and were then treated during 7 days with i) vancomycin 50 mg/kg (n=4), ii) vancomycin 50 mg/kg and taurocholate 20 mg/kg (n=6) and iii) vancomycin 50 mg/kg, nystatin 8,500 IU/kg and sodium taurocholate 20 mg/kg (n=5). After finishing the treatment, mice were evaluated daily until day 18 when they were sacrificed.

FIG. 26 shows the average weight of the mice of the experiment indicated in FIG. 25. Shown is the weight in the R-CDI period (after discontinuing treatment). Indicated are mice of the groups treated with i) vancomycin, ii) vancomycin and nystatin 8,500 IU/kg and iii) vancomycin 50 mg/kg, nystatin 8,500 IU/kg and sodium taurocholate 20 mg/kg, according to the experimental design of FIG. 25. No significant differences were observed in the weight of the different groups.

FIG. 27 shows the time taken by mice infected by C. difficile and treated with i) vancomycin, ii) vancomycin and nystatin 8,500 IU/kg and iii) vancomycin 50 mg/kg, nystatin 8,500 IU/kg and sodium taurocholate 20 mg/kg (according to the experimental design of FIG. 25), to present diarrhea associated to a recurrent C. difficile infection.

### Example 8

Treatment of CDI with a Formulation Comprising Nystatin, Vancomycin and Ramoplanin Reduces Cases of Diarrhea in R-CDI.

We studied the effect of the administration of a pharmacological formulation comprising nystatin, vancomycin and additionally ramoplanin after manifestation of the symptoms of CDI. We found that the pharmacological formulation of nystatin, vancomycin and ramoplanin reduces the cases of diarrhea caused by R-CDI.

Since in the results of the previous examples, it was observed that a formulation comprising nystatin and vancomycin serves for reducing the incidence of R-CDI, the same as in example 7, seeking to increase the efficiency of the formulation, ramoplanin was added because this antibiotic is capable of binding to the exosporium of the C. difficile spore, inactivating it when it germinates.

In this sense, we administered a formulation comprising 17,000 IU/kg of nystatin (as observed in example 6), 50 mg/kg of vancomycin and 8 mg/kg of ramoplanin and, as a control treatment, 17,000 IU/kg of nystatin and 50 mg/kg of vancomycin. For the purpose of this study, animals that did not manifest severe CDI were discarded from the analysis.

We observed that when administering the formulation of nystatin, vancomycin and ramoplanin, no significant differences were observed in the manifestation of diarrhea during R-CDI (FIG. 28).

Of the mice treated with nystatin, vancomycin and ramoplanin, 57% manifested diarrhea in R-CDI, while in the control group, 100% manifested diarrhea (FIG. 28).

FIG. 21 shows a scheme of experimental design for evaluation of R-CDI. However, for this example, 2 formulations were evaluated: i) nystatin+vancomycin (n=5) and ii) nystatin+vancomycin+ramoplanin (n=7).

1. A formulation for treating or preventing the risk of developing recurrent C. difficile infections in a subject, wherein the formulation comprises one or more antibiotics having activity against C. difficile and an agent that inhibits internalization of C. difficile spores.

2. The formulation of claim 1, wherein the one or more antibiotics are selected from the group consisting of vancomycin, ramoplanin, metronidazole, fidaxomicin and rifaximin.

3. The formulation of claim 1, wherein the one or more antibiotics comprise vancomycin and/or ramoplanin.

4. (canceled)

5. The formulation of claim 1, wherein the agent that inhibits internalization of C. difficile spores is nystatin.

6. The formulation of claim 1, wherein the one or more antibiotic is vancomycin and the agent that inhibits internalization of C. difficile spores is nystatin.

7. The formulation of claim 1, further comprising a spore germinant.

8. The formulation of claim 7, wherein the spore germinant is taurocholate.

9. The formulation of claim 1, further comprising a pharmaceutically acceptable solvent or carrier.

10. The formulation of claim 1, further comprising one or more pharmacologically acceptable excipients.

11. The formulation of claim 1, wherein the recurrent C. difficile infection is C. difficile colitis.

12. The formulation of claim 11, wherein the C. difficile colitis is pseudomembranous colitis.

13. (canceled)

14. The formulation of claim 1, wherein the formulation is manufactured in the form of syrup, capsules, serum, granules, encapsulated in nanoparticles.

15. The formulation of claim 1, wherein the one or more antibiotics having activity against C. difficile and the agent that inhibits internalization of C. difficile spores have a weight ratio of about 40:1 to about 3:1.

16. The formulation of claim 1, wherein the one or more antibiotics having activity against C. difficile and the agent that inhibits internalization of C. difficile spores have a weight ratio of about 9:1.

17. The formulation of claim 1, wherein the one or more antibiotics having activity against C. difficile and the agent that inhibits internalization of C. difficile spores have a weight ratio of about 4:1.

18. The formulation of claim 1, wherein the one or more antibiotics having activity against C. difficile are present in an amount that ranges from about 100 mg to about 4 g per day.

19. The formulation of claim 1, wherein the one or more antibiotics having activity against C. difficile are present in an amount of about 50 mg/kg/day.

20. The formulation of claim 1, wherein the agent that inhibits internalization of C. difficile spores is present in an amount that ranges from about 100,000 UI to about 3,000,000 UI per day.

21. The formulation of claim 1, wherein the agent that inhibits internalization of C. difficile spores is present in an amount that ranges from about 4,250 UI/kg to about 17,000 UT/kg.

22. The formulation of claim 21, wherein the agent that inhibits internalization of C. difficile spores is nystatin and it is present in an amount of 8,500 UI/kg.

23. The formulation of claim 8, wherein taurocholate is present in an amount of 20 mg/kg.

24-47. (canceled)

48. A method for treating or preventing the risk of developing recurrent C. difficile infections in a subject in need thereof, comprising administering to the subject an effective amount of one or more antibiotics having activity against C. difficile and an agent that inhibits internalization of C. difficile spores.

49. The method of claim 48, wherein the one or more antibiotics are selected from the group consisting of vancomycin, ramoplanin, metronidazole, fidaxomicin, rifaximin.

50. The method of claim 48, wherein the one or more antibiotics comprise vancomycin and/or ramoplanin.

51. (canceled)

52. The method of claim 48, wherein the agent that inhibits internalization of C. difficile spores is nystatin.

53. The method of claim 48, wherein the one or more antibiotic is vancomycin and the agent that inhibits internalization of C. difficile spores is nystatin.

54. The method of claim 48, further comprising a spore germinant.

55. The method of claim 54, wherein the spore germinant is taurocholate.

56. The method of claim 48, wherein the recurrent C. difficile infection is C. difficile colitis.

57. The method of claim 56, wherein the C. difficile colitis is pseudomembranous colitis.

58. (canceled)

59. The method of claim 48, wherein the one or more antibiotics having activity against C. difficile and the agent that inhibits internalization of C. difficile spores are administered at a weight ratio of about 40:1 to about 3:1.

60. The method of claim 48, wherein the one or more antibiotics having activity against C. difficile and the agent that inhibits internalization of C. difficile spores are administered at a weight ratio of about 9:1.

61. The method of claim 48, wherein the one or more antibiotics having activity against C. difficile and the agent that inhibits internalization of C. difficile spores are administered at a weight ratio of about 4:1.

62. The method of claim 48, wherein the effective amount of the one or more antibiotics having activity against C. difficile ranges from about 100 mg to about 4 g per day.

63. The method of claim 48, wherein the one or more antibiotics having activity against C. difficile are administered in an amount of about 50 mg/kg/day.

64. The method of claim 48, wherein the effective amount of the agent that inhibits internalization of C. difficile spores ranges from about 100,000 UI to about 3,000,000 UI per day.

65. The method of claim 48, wherein the agent that inhibits internalization of C. difficile spores is administered in an amount that ranges from about 4,250 UI/kg to about 17,000 UI/kg.

66. The method of claim 65, wherein the agent that inhibits internalization of C. difficile spores is nystatin and it is administered in an amount of 8,500 UI/kg.

67. The method of claim 55, wherein taurocholate is administered in an amount of 20 mg/kg.

68-92. (canceled)

93. A kit for treating or preventing the risk of developing recurrent C. difficile infections in a subject in need thereof, comprising:
(a) one or more antibiotics having activity against C. difficile and a package insert comprising instructions for using the one or more antibiotics in combination with an agent that inhibits internalization of C. difficile spores;
(b) one or more antibiotics having activity against C. difficile and an agent that inhibits internalization of C. difficile spores and a package insert comprising instructions for using the one or more antibiotics and the agent that inhibits internalization of C. difficile spores; or
(c) an agent that inhibits internalization of C. difficile spores and a package insert comprising instructions for using the agent that inhibits internalization of C. difficile spores in combination with one or more antibiotics having activity against C. difficile.

94. (canceled)

95. (canceled)

96. The kit of claim 93, wherein the one or more antibiotics are selected from the group consisting of vancomycin, ramoplanin, metronidazole, fidaxomicin and rifaximin.

97. The kit of claim 93, wherein the one or more antibiotics comprise vancomycin and/or ramoplanin.

98. (canceled)

99. The kit of claim 93, wherein the agent that inhibits internalization of C. difficile spores is nystatin.

100. The kit of claim 93, wherein the one or more antibiotic is vancomycin and the agent that inhibits internalization of C. difficile spores is nystatin.

101. The kit of claim 93, further comprising a spore germinant.

102. The kit of claim 101, wherein the spore germinant is taurocholate.

103. The kit of claim 93, wherein the recurrent C. difficile infection is C. difficile colitis.

104. The kit of claim 103, wherein the C. difficile colitis is pseudomembranous colitis.
